(19) United States
(12) Patent Application Publication
(10) Pub. No.: US 2006/0247195 A1
(43) Pub. Date: Nov. 2, 2006

(54) Method of altering cell properties by administering rna

(75) Inventor: Stephen Ray, Oxfordshire (GB)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Ribostem Limited, Oxford (GB)

(21) Appl. No.: 10/563,897

(22) PCT Filed: Jul. 9, 2004
(86) PCT No.: PCT/GB04/02981

§ 371 Date: Jun. 8, 2006

(30) Foreign Application Priority Data
Jul. 9, 2003 (GB) 0316089.2

(51) Int. Cl. A61K 48/00

(52) U.S. Cl. 514/44

(57) ABSTRACT

The invention relates to the alteration of cell properties using RNA molecules. In particular, it relates to the alteration of the ability of cells to mobilise, migrate, integrate, proliferate and/or differentiate. For example, it relates to the induction of differentiation of stem cells, including the acquisition of the ability to migrate, integrate, and proliferate. It also relates to the induction of in vivo stem cell mobilisation, migration, integration, proliferation and/or differentiation. Accordingly, it relates to the promotion of stem cell-mediated functional repair. The invention also relates to the reversal of differentiation of differentiated cells. All these effects may be effected by providing isolated RNA comprising a RNA sequence extractable from cells comprising the desired cell type(s) to a population of cells under conditions whereby the alteration of the cell property is achieved.

All documents cited herein are incorporated by reference in their entirety.

### FIELD OF THE INVENTION

The present invention relates to the alteration of cell properties. In particular, it relates to the alteration of one or more of the capacities of a cell to mobilise, migrate, integrate, proliferate and differentiate, where such capacity is latent or evident, and where each capacity may be manifested in any order. For example, it relates to the alteration of properties of stem cells, including the acquisition of the evident or latent capacity to mobilise, migrate, integrate, proliferate and differentiate. It also relates to the in vivo alteration of stem cell properties, including the acquisition of the evident or latent capacity to mobilise, migrate, integrate, proliferate and differentiate. It also relates to the in vitro alteration of the stem cell properties, including the acquisition of the capacity to mobilise, migrate, integrate, proliferate and differentiate, where such alterations of property may be evident or latent in vitro, or may only become evident after subsequent introduction to a host in vivo, or after subsequent introduction into a further phase of in vitro culture, including introduction into an ex vivo preparation. Accordingly, it relates to the promotion of functional repair and or regeneration. The invention also relates to the alteration of the genotype of a cell, both in vitro and in vivo. The invention further relates to the induction of differentiation of stem cells, and to the reversal of differentiation of differentiated cells.

### BACKGROUND OF THE INVENTION

Stem cells, and their application in regenerative medicine continue to dominate the scientific and lay press. Stem cells are undifferentiated cells that are capable of both self-renewal and also differentiation into one or more differentiated cell types. This dual ability to both divide to produce further stem cells and to differentiate means that stem cell populations can maintain their number whilst also giving rise to a large number of differentiated cells.

Stem cells reside in numerous locations of both plants and animals. Although much of the initial work carried out on stem cells focussed on embryonic stem cells, adult tissues also contain stem cells. Stems cells play essential roles such as in normal tissue repair. Stem cells also give rise to differentiated cells to replace those lost during the normal functioning of the body. For example, haematopoietic stem cells differentiate to give rise to various progenitor cells that in turn give rise to the various cells of the immune system. Thus as mature immune cells die they are replaced by new immune cells originating from the haematopoietic stem cells. In an analogous fashion, certain types stem cells may give rise other types of stem cells.

Stem cell populations can be routinely isolated for culture outside of the body or can be manipulated in vivo. Stem cells may be isolated from a variety of sources including normal adult tissues, pre-implantation embryos, foetal tissues (at various stages of development) and tumours. Both adult and embryonic stem cell lines have been established. Stem cell lines may be maintained in culture more-or-less indefinitely. Stem cell lines can also be manipulated in culture to introduce specific genetic modifications using techniques such as gene targeting. Stem cell populations cultured or manipulated in vitro may then be introduced to a host in vivo, or into a subsequent form of tissue culture.

Central to any application of stem cell technology is the ability to control the capacities of a stem cell to mobilise, migrate, integrate, proliferate and differentiate both in vitro and after transplantation to recipient humans, animals and plants. Although the ability of stem cells in vivo to mobilise, migrate, integrate, proliferate and differentiate is well known, the ability to artificially control the mobilisation, migration, integration, proliferation and differentiation of stem cells into mature cells in target tissues is still in its infancy. Existing methods largely rely on exposing cultured stem cells to specific growth factors and/or growth conditions. Only a relatively limited number of specific differentiated cell types can be produced using such methods.

WO 95/12665 discloses a method for differentiating embryo stem cells into desired cell lines. The embryonic stem cells are engineered with DNA encoding a protein or polypeptide that promotes differentiation of the stem cells into a specific cell line. The DNA may encode a transcription factor found in the particular cell line.

Dai et al. (2000) showed that erythropoiesis in differentiating embryoid bodies could be enhanced by retrovirus-mediated gene transfer of a human erythropoietin receptor gene into murine embryonic stem cells.

WO 01/00650 discloses methods for de-differentiating recipient cells (e.g. human somatic cells) by the introduction of cytoplasm from less a differentiated cell type (e.g. an oocyte).

Tada et al. (2001) demonstrated that the fusion of adult thymocytes with embryonic stem cells could reset certain aspects of the epigenotype of the somatic cells to those of the embryonic stem cells. For example, the hybrids showed pluripotency in vivo.

### SUMMARY OF INVENTION

The present invention is concerned with the alteration of cell properties. In particular, it relates to the alteration of differentiation and the ability of cells to mobilise, migrate, integrate, proliferate and differentiate. The invention is also concerned with the alteration of the genotype of a cell and with the control of differentiation.

Without wishing to be bound by theory, the invention is based on two hypotheses: (a) the behaviour of cells used for tissue regeneration is governed by the transfer of information via RNA from tissues in need of regeneration to effector cells; and (b) alteration of the genotype of a cell may be effected by the transfer of information from one cell to another via RNA.

Accordingly, the present invention is concerned with the promotion of stem cell-mediated functional repair, the treatment of various disease conditions by influencing mobilisation, migration, integration, proliferation and differentiation of cells, the differentiation of adult cells and stem cells in general and their acquisition of the ability to mobilise, migrate, integrate, proliferate and differentiate. The invention is also concerned with the alteration of the genotype of a cell, and the treatment of various disease conditions by alteration of the genotype of cells. The present inventors have found that it is possible to induce stem cells to differentiate into a desired differentiated cell type and conversely that it is possible to reverse the differentiation of differentiated cells to provide stem cells. The inventors have also found that it is possible to induce stem cells to mobilise, migrate, integrate, proliferate and differentiate into a desired differentiated cell type which is integrated into a targeted tissue, that it is possible to reverse the differentiation of differentiated cells to provide stem cells, and that it is also possible to alter the genotype of a cell. This is achieved by providing specific RNA sequences to the target cells.

The ability to influence cell fate or genotype allows a variety of clinically useful phenomena to be induced including allowing diseased cells, tissue and organs to be repaired, allowing the genetic constitution of cells to be altered, allowing specific cell types and cell fates to be induced, allowing immunological profiles to be changed at will, allowing the induction of particular immune functions and so on. The ability to induce stem cell mobilisation, migration, integration, proliferation and differentiation in vivo means that stem cell-mediated functional repair and genotype alteration may be beneficially promoted in intact organisms, and particularly animals.

Accordingly, the present invention provides a method for altering a cell property towards a property of one or more desired cell types comprising providing isolated RNA comprising a RNA sequence extractable from cells comprising said desired cell type(s) to a population of cells under conditions whereby the alteration of the cell property of said cells is achieved.

The isolated RNA may be extractable from or extracted from one or more cell types that possess the property or properties of interest. The isolated RNA may comprise the sequence of RNA extractable from one or more cell types that possess the property or properties of interest. It is thus not always necessary to extract RNA from the desired cell types; the RNA sequence conferring the advantageous property or properties onto the cell type may be generated synthetically, for example, using a recombinant expression system. Larger quantities of the desired RNA may be produced by the in vitro expansion of isolated RNA.

The population of cells may be exposed to the RNA in vitro, or in vivo. In vitro, the population of cells may for example be a cell culture, such as in a cell culture dish or roller bottle or cells growing on a support, membrane, implant, stent or matrix; or a tissue, such as an isolated tissue grown outside the body. In vivo, the population of cells may be an organism, such as a human patient, or a tissue isolated from an organism, such as an organ, a specific part of an organ, or a specific cell type or collection of cell types.

The method of the invention may be used to improve stem cell-mediated repair, either in vivo or in vitro. In one embodiment, this aspect of the present invention provides a method of inducing totipotent or pluripotent stem cells of a stem cell line or derived from a tissue of an animal or plant to differentiate into one or more desired cell types, which comprises providing isolated RNA comprising RNA extractable from tissue or cells comprising said desired cell type(s) to a cell culture of said stem cells under conditions whereby the desired differentiation of said stem cells is achieved. The cells generated in vitro in this manner may then be delivered into a recipient. For in vivo treatment, the stem cells may reside and be exposed to the RNA in situ, in the body of the patient. Alternatively, the stem cells, with or without pre-treatment as above, may be administered to a tissue or an organism on their own or in conjunction with RNA according to the invention, to induce mobilisation, migration, integration, proliferation and differentiation of stem cells in vivo in the treated subject. Cells and RNA may also be administered in simultaneous, separate or sequential application with other therapies effective in treating a particular disease. In one embodiment, RNA extractable from one or more stem cell types or stem cell active tissue(s) may be administered in simultaneous, separate or sequential application optionally with cells, such as stem cells. For example, in a specific preferred embodiments, RNA from an embryo or foetus, from the whole body, an organ, a specific part of an organ, or a specific cell type or collection of cell types is administered in simultaneous, separate or sequential application with stem cells, or cells derived from in vitro treatment of stem cells, particularly bone marrow stem cells.

In some cases the isolated RNA itself may be used to induce differentiation in situ. Similarly, the isolated RNA itself may be used to induce mobilisation, migration, integration, proliferation and differentiation in situ. Similarly, the isolated RNA itself may be used to induce genotypic modification in situ. Thus in another aspect the invention also provides for the use of the RNA capable of inducing differentiation of cells, particularly stem cells, in the treatment of, or in the manufacture of a medicament for use in improving or rectifying, tissue or cellular damage or a genetic disease. The invention also provides for the use of the RNA capable of inducing migration, mobilisation, integration, proliferation and differentiation of cells, particularly stem cells, in the treatment of, or in the manufacture of a medicament for use in improving or rectifying, tissue or cellular damage or a genetic disease, including repair of diseased cells, induction of specific cell types and cell fates, changing the immunological profiles of cells, and inducing particular desired immune functions or properties. The invention also provides for the use of the RNA capable of inducing genotypic modification of cells, particularly stem cells, in the treatment of, or in the manufacture of a medicament for use in improving or rectifying, tissue or cellular damage or a genetic disease, including repair of diseased cells, alteration of the genetic constitution of cells, induction of specific cell types and cell fates, changing the immunological profiles of cells, and inducing particular desired immune functions or properties. The isolated RNA may be provided to the cell population as a medicament in which the RNA forms the principal active ingredient of the medicament.

The isolated RNA may be used to induce mobilisation, migration, integration, proliferation and differentiation of stem cells in vivo. Accordingly, in another aspect the invention provides a method of treatment comprising administration of the RNA capable of inducing differentiation of stem cells in a therapeutically effective amount to a patient in need thereof. The invention also provides a method of treatment comprising administration of the RNA capable of inducing mobilisation, migration, integration, proliferation and differentiation of stem cells in a therapeutically effective amount to a patient in need thereof. Such methods may be used, for example, to promote stem cell-mediated functional repair, including repair of diseased cells, alteration of the genetic constitution of cells, induction of specific cell types and cell fates, changing the immunological profiles of cells, and inducing particular desired immune functions or properties.

Isolated RNA comprising RNA extractable from particular desired type(s) of stem cell or stem cell line may thus be used to promote stem cell-mediated functional repair in vivo, and to improve or rectify tissue or cellular damage or a genetic disease. Such damage may be due to, for example, disease, age or genetic makeup or genetic mutation, trauma, surgery, any other form of treatment, disease, or accidental or intentional morbidity.

Alternatively, the RNA may be applied to the cell population in conjunction with other active agents, including, for example, stem cells, or cells derived in vitro from stem cells according to a method as described above. The RNA and other active agents may be administered simultaneously, sequentially or separately.

The method of the invention may be used to induce stem cells to mobilise, migrate, integrate, proliferate and/or differentiate into one or more desired cell types. This aspect of the present invention provides a method of inducing totipotent or pluripotent stem cells of a stem cell line or derived from a tissue of an animal or plant to mobilise, migrate, integrate, proliferate and/or differentiate into one or more desired cell types, which comprises providing isolated RNA comprising RNA extractable from tissue or cells comprising said desired cell type(s) to a cell culture of said stem cells under conditions whereby the desired mobilisation, migration, integration, proliferation and/or differentiation of said stem cells is achieved. In some embodiments, the totipotent or pluripotent stem cells are treated in vitro. In other embodiments, the totipotent or pluripotent stem cells are treated in vivo, in the body of the patient. The stem cells employed may be adult stem cells.

The method of the invention may also be used to induce adult cells to mobilise, migrate, integrate, proliferate and/or differentiate into one or more desired stem cell types. In this aspect, the invention thus provides a method for obtaining stem cells. Thus the invention provides a method of reversing in vitro the differentiation of differentiated cells of a cell line or obtained from the tissue of an animal or a plant to produce a desired type or types of totipotent or pluripotent stem cell(s) or stem cell line(s), which comprises providing isolated RNA comprising RNA extractable from the desired type(s) of stem cell or stem cell line to a cell culture of said differentiated cells whereby the desired reversal of differentiation of the differentiated cells into said type(s) of stem cell or stem cell line type(s) is achieved. The invention provides stem cells obtained using such methods. The invention also provides for the use of such cells in the manufacture of a medicament for use in improving or rectifying tissue or cellular damage or a genetic disease. In a further aspect the invention provides a method of treatment comprising administration of such cells in a therapeutically effective amount to a patient in need thereof.

In some embodiments, the stem cells employed are adult stem cells and the desired cell type(s) comprise embryonic stem cells or embryonic stem cell lines. Accordingly, the present invention also provides a method of producing embryonic stem cells or embryonic stem cell-like cells from adult stem cells.

More generally, the stem cells employed may be from a post-embryonic developmental stage e.g. foetal, neonatal, juvenile, or adult, or any sub-stage within these stages, and the desired cell type(s) comprise embryonic stem cells or embryonic stem cell lines. The invention thus provides a method of producing embryonic stem cells or embryonic stem cell-like cells from such post-embryonic stage cells. For example, the invention allows the generation of stem cells with properties of embryonic stem cells for the treatment of an adult, by derivation from autologous (adult) stem cells from that same individual.

Certain types of stem cell are present only at particular developmental stages (e.g. there is a certain type of erythrocyte-producing stem cell present in the liver of a certain stage of foetal development, and liver stems cells with this type of behaviour are not present in the adult). Accordingly, the invention provides a method of producing stem cells or stem cell-like cells which are present only at certain specific stages of development, from stem cells available at other stages of development.

In some embodiments of the invention, the stem cells employed are stem cells of a particular type (e.g. a bone marrow mesenchymal stem cell) and the desired cell type comprises a different type of stem cells (e.g. neural stem cells). The present invention provides a method of producing cells with the properties of one type of stem cell from another type of stem cell.

Any particular type of stem cell may have different capacities depending on the developmental stage of the natural host. In some embodiments, therefore, the stem cells employed are stem cells of a particular type and of a particular developmental stage (e.g. bone marrow mesenchymal stem cells from an adult) and the desired cell type(s) comprise the same or different type of stem cells with the properties pertaining to those of a different developmental stage (e.g. bone marrow mesenchymal stem cells of a neonate). Accordingly, the invention also provides a method of producing cells with properties pertaining to a particular type of stem cells from one development stage from stem cells of the same or different type from a different developmental stage.

The ability to produce differentiated cells from stem cells means that large numbers of desired differentiated cells can be obtained. In addition, the ability to produce stem cells from differentiated cells offers a much simpler, less labour intensive and less invasive way to provide stem cells in comparison to methods where stem cells have to be isolated directly. It also means that highly pluripotent stem cells may be obtained from adult tissues, making the use of embryonic tissues unnecessary. By combining the techniques for producing stem cells with those for differentiating them, large numbers of desired differentiated cells can be produced. The methods and medicaments of the invention mean that differentiation can be controlled both in tissue culture and in intact organisms, and particularly animals.

The method of the invention may also be used to induce adult cells to differentiate into other, different adult cell types. Examples of such differentiation include repair of diseased (including cancerous) cells, alteration of the genetic constitution of cells, induction of specific cell types and cell fates, changing the immunological profiles of cells, and inducing particular desired immune functions or properties.

The invention also provides cells obtained by the above methods. Such differentiated cells may be used in the manufacture of medicaments for treating a number of disorders. Thus, in a further aspect the invention provides for the use of the cells in the manufacture of a medicament for use in improving or rectifying tissue or cellular damage or degeneration or a genetic disease. The invention includes methods of treatment that comprise administration of these cells in a therapeutically effective amount to a patient in need thereof. Furthermore, the differentiated cells may be used for diagnostic and/or research purposes and/or in the manufacture of reagents used for diagnosis and/or research. Thus, in a further aspect, the invention provides for the use of the cells in diagnosis or research and in the manufacture of a reagents for diagnosis or research.

The stem cells obtained using the methods of the invention may be induced to mobilise, migrate, integrate, proliferate and/or differentiate using a method in accordance with the invention. Thus in a further aspect the invention provides a method of producing differentiated cells, which comprises: (a) performing a method in accordance with the invention to produce stem cells or a stem cell line from differentiated cells; (b) performing a method in accordance with the invention on the stem cells or stem cell line to produce differentiated cells. The method may be performed in vivo and/or in vitro.

The invention also provides cells obtained by such methods. The cells obtained may be used to treat a number of diseases. Thus the invention also provides for the use of such cells in the manufacture of a medicament for use in improving or rectifying tissue or cellular damage or a genetic disease and for the use of such cells for diagnosis or research. In a further aspect, the invention provides a method of treatment comprising administration of such cells in a therapeutically effective amount to a patient in need thereof.

In some cases desired genetic modifications may be introduced into the stem cells, or cells derived from the stem cells by the method.

### BRIEF DESCRIPTION OF THE DRAWINGS

FIG. 1: The effects of brain RNA differentiated stem cells on age-related damage to the rat brain assessed by spatial learning and memory performance of recipient animals. Ex-breeder male rats aged between 468 to 506 days were given intravenously either untreated bone marrow stem cells or bone marrow stem cells treated with brain RNA extract. The results for control rats that received untreated stem cells (closed boxes) and those for experimental rats that received brain treated stem cells (open circles) are shown. The results show a remarkable increase in learning ability in the experimental rats.

FIG. 2: The effects of spine RNA differentiated stem cells on an animal model of motor neurone disease. SOD 1 mice were given intravenously either bone marrow stem cells treated with spine RNA extract, untreated bone marrow stem cells or physiological saline. The results for experimental mice that received spine RNA-treated stem cells (closed boxes), control mice that received untreated stem cells (open triangles) and control mice that received physiological saline (closed circles) are shown. The results show that pre-treatment of stem cells with spine derived RNA dramatically improved the efficacy of stem cell treatment in an established model of progressive neurodegenerative disease.

FIG. 3: The influence of donor tissue developmental stage on the effect of brain RNA differentiated stem cells on age related damage to the mouse brain assessed by spatial learning and memory performance of recipient animals. 254-299 day old C57/B1 mice were given intravenously either bone marrow stem cells treated with foetal (E15) brain RNA extract, bone marrow stem cells treated with adult (90 day) brain RNA extract or untreated bone marrow stem cells. The results for control mice that received untreated stem cells (closed boxes), experimental mice that received foetal brain treated stem cells (closed circles) and experimental mice that received adult brain treated stem cells (open triangles) are shown. The results show an increase in learning ability in the experimental mice, with the mice that received foetal brain treated stem cells demonstrating significantly faster learning.

FIG. 4: The effects of direct injection of bone marrow stem cell derived RNA on age related damage to the rat brain assessed by spatial learning and memory performance of recipient animals. Ex-breeder male rats aged between 433 to 570 days were given injections of either bone marrow stem cell RNA or bone marrow stem cell RNA treated with RNaze into the right lateral ventricle. The results for control rats that received RNaze treated stem cell RNA (closed boxes) and those for experimental rats that received stem cell RNA (open circles) are shown. The results show that control rats could not learn the task, while the stem cell RNA treated animals could learn the task with comparable performance to young rats.

### DETAILED DESCRIPTION

The inventors have found that provision of RNA sequences from particular sources to cells can influence cell properties. Accordingly, the present invention is in one aspect concerned with the promotion of stem cell-mediated functional repair. By “repair” is meant restoration, regeneration, strengthening, renewal, rejuvenation, or partial or complete regrowth or renewal of a tissue. Stem cell-mediated repair may occur either in vitro or in vivo. The invention is also concerned with: the differentiation of stem cells to adult, specialised cells; the differentiation of adult specialised cells to stem cells; and the differentiation of specialised adult cells to other adult cells of different specialties. This is achieved by providing specific RNA sequences to the target cells. The invention is also concerned with the modification of the genotype of a cell, in vitro or in vivo.

The present invention is generally concerned with the alteration of a cell property. By “property” is meant any desired property of a cell, including a biological property that is reflective of the type of biological molecule(s) that is present in or on the surface of, or secreted by, a cell. A desired property includes the active state of a particular biological molecule(s) in the cell, or the capability possessed by a cell for a particular behaviour.

The property may be a latent property or may be evident in the cell. The property may be a particular phenotype, by which is meant any observable physical or biochemical characteristic. Such a phenotype change may be, for example, the expression of a cell surface marker, altered immune function, altered MHC restriction, altered activity of one or more proteins, and so on. A desired phenotypic change may be more extreme e.g. redirection of cell function from one tissue to another, such as a liver cell towards a kidney cell. Such a phenotypic change may be reversal of tumour cell activity toward healthy cell activity.

The property may thus be any desired function that is possessed by a cell. The term “function” is meant to include any biological activity that is effected by the desired cell type. Examples of functions include those that are specific to a particular tissue, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, slain, and gastrointestinal tract, lung, bone, cartilage, tendon, hair follicle, sense organ (for example, ear, eye), any gland either endocrine, exocrine, paracrine, such as thyroid, thymus, pituitary, adrenal, pancreatic, reproductive system (for example, testicular, prostate, seminal vesicle, ovarian, uterine, fallopian mammary), dental, vascular, digestive tract tissues (for example, stomach, gall bladder, intestines, colon). At a more detailed level, the function of particular cell types within a tissue type may be of interest, for example within brain tissue, neuronal cells or cortical neurones or glial cells have more specialised functions within the brain. At a more detailed level still, desired functions may be at a molecular level, where it is desired for specific molecules to be expressed on the surface of cells, such as specific T cell receptors in the case of T cells of the immune system. It is not possible for any list of desired function to be exhaustive and equivalent functions that may be desired in each circumstance will be apparent to the skilled reader.

The alteration of the property may result in the cell undergoing differentiation towards a more specialized form or function, for example from a stem cell towards an adult cell with a specialised function (for example, a hepatocyte). The alteration may also result in the cell undergoing reverse differentiation towards a less specialized form or function, for example from an adult specialised cell towards a stem cell. The alteration may also result in the cell and its progeny acquiring the behaviour of mobilisation, migration towards, and/or integration with, one or more tissues, organs or other sites, and proliferation. By “mobilisation” is meant change of stem cells from a quiescent resting state and, when in vivo, departure from their quiescent resting location. By “migration” is meant movement of stem cells from their point of mobilisation or artificial delivery, towards and into a target tissue. By “integration”, is meant the interaction of stem cells and their integration with a target cell populations and environment.

The alteration may also result in the cell and its progeny acquiring the behaviour of proliferation, prior to, during, or after migration and integration. By “proliferation”, is meant division of cells and their progeny to provide new tissue.

The alteration may also result in the cell undergoing a genetic transformation so as to acquire an altered, inheritable genotype. Such an altered genotype may reverse a genetic mutation that a cell has acquired through somatic mutation or which the cell has inherited. In this way, genetic disease may be treated or prevented. Such an altered genotype may provide a genetic change which provides for an additional, modified, removed or disabled function. This method of transformation is a form of gene therapy whereby a cell is genetically altered, preferably inheritably so that the alteration is passed to any progeny. Accordingly, the methods of the present invention can be used in gene therapy, either of somatic or germ line cells, for the provision of cells that are genetically altered, preferably inheritably. Further examples will be clear to those of skill in the art.

Accordingly, in one aspect the present invention provides a method of directing differentiation of stem cells towards one or more desired cell types comprising providing isolated RNA comprising a RNA sequence extractable from cells comprising said desired cell type(s) to a population of stem cells under conditions whereby the desired differentiation of stem cells in said population is achieved. The invention also provides a method of directing mobilisation, migration, integration, proliferation and differentiation of stem cells towards one or more desired cell types in vitro, or integrated with a target tissue in vivo, comprising providing isolated RNA comprising a RNA sequence extractable from cells comprising said desired cell type(s) to a population of stem cells under conditions whereby the desired differentiation of stem cells in said population is achieved. The RNA may be extractable or extracted from cells comprising said desired cell type(s).

This method allows the direction of differentiation to be dictated towards a particular speciality. For example, the stem cells may be directed towards liver function, or more specifically hepatocyte function. The method allows the mobilisation of a particular type of stem cell, for example bone marrow mesenchymal stem cells. The method allows for migration and integration into a particular tissue e.g. the left femur.

The invention provides methods and medicaments for the controlled manipulation of any cell, in particular stem cells, to induce the cell to differentiate into a desired differentiated cell type. Such methods include the improvement of stem cell-mediated repair, through directing the mobilisation, migration, integration, proliferation and differentiation of stem cells.

Such methods include the induction of stem cells to differentiate into one or more desired adult cell types. In addition, the invention also provides methods for inducing the reversal of differentiation of a differentiated cell to provide a stem cell. The two methods may be combined so that, for example, a stem cell can be obtained from a differentiated cell and then differentiated to provide differentiated cells of a different or same cell type. Prior to the latter differentiation, the stem cells may be expanded in number and/or manipulated in a desired fashion for example to introduce a desired genetic modification.

A stem cell may, for example, be induced to differentiate in order to achieve a specific terminal differentiated state. Using the methods of the invention it is also possible to ensure that the differentiated cells are immunologically compatible with the intended recipient. The ability to choose what type of cell to induce the stem cell to differentiate into means that it is possible to produce a variety of different cell types from a single stem cell line or stem cell line. The RNA molecules of the invention, or differentiated cells types obtained, may be employed in treating, or in the manufacture of medicaments for treating, various disorders. In particular they may be used for improving or rectifying tissue or cellular damage or a genetic disease. The invention also provides methods and medicaments for the induction of in vivo stem cell mobilisation, migration, integration, proliferation and/or differentiation and the promotion of stem cell-mediated functional regeneration and/or repair.

Such methods include: the induction of stem cells of one cell type to differentiate into one or more other desired stem cell types; the induction of adult cells to differentiate into one or more desired stem cell types and the induction of adult cells to differentiate into other, different adult cell types.

The ability to influence cell fate using RNA allows diseased cells to be repaired, allows the genetic constitution of cells to be altered, allows specific cell types and cell fates to be induced, allows immunological profiles to be changed at will, allows the induction of particular immune functions and so on.

Stem Cells

The invention may be used to produce or differentiate any suitable stem cell. The invention may also be used to induce in vivo stem cell mobilisation, migration, integration, proliferation and differentiation. A stem cell is generally understood to be a cell capable of self-renewal that is also capable of differentiation into one or more specific differentiated cell type(s). Stem cells may be pluripotent, that is they may be capable of giving rise to a plurality of different differentiated cell types. In some cases the stem cells may be totipotent, that is they may be capable of giving rise to all of the different cell types of the organism that they are derived from. The invention is applicable to pluripotent stem cells or totipotent stem cells.

In a particularly preferred embodiment the invention is used to differentiate or obtain adult stem cells. Stem cells are known to occur in a number of locations in the animal body. Stem cells differentiated or obtained by the present invention may be those from any of the organs and tissues in which stem cells are present. Examples include stem cells from the bone marrow, haematopoietic system, neuronal system, the brain, muscle stem cells or umbilical cord stem cells. The stem cells may in particular be bone marrow stromal stem cells, neuronal stem cells or haematopoietic stem cells, in a preferred case they may be bone marrow stromal stem cells or neuronal stem cells. In particular when the methods of the invention are used to induce differentiation of a stem cell, the stem cell is a bone marrow stromal cell.

The stem cells may be plant or animal stem cells.

In a preferred case, the stem cells will be animal stem cells and preferably mammalian stem cells. In one preferred embodiment, the stem cells may be human stem cells. Alternatively, the stem cells may be from a non-human animal and in particular from a non-human mammal. The stem cells may be those of a domestic animal or an agriculturally important animal. The animal may, for example, be a sheep, pig, cow, horse, bull, or poultry bird or other commercially-farmed animal. The animal may be a dog, cat, or bird and in particular from a domesticated animal. The animal may be a non-human primate such as a monkey. For example, the primate may be a chimpanzee, gorilla, or orangutan. The stem cells may be rodent stem cells. For example, the stem cells may be from a mouse, rat, or hamster.

In another preferred case, the stem cells will be plant stem cells. Stem cells are known to occur in a number of locations in the seed and developing or adult plant. Stem cells differentiated or obtained in the present invention may be those from any of the tissues in which stem cells are present. Examples include stem cells from the apical or root meristems. In one preferred embodiment, the stem cells are from an agriculturally important plant. The plant may, for example, be maize, wheat, rice, potato, an edible fruit-bearing plant or other commercially farmed plant.

In many cases the differentiated cells may be intended to treat a subject, and in the manufacture of medicaments. In such cases the stem cells may be from the intended recipient. This may particularly be the case where the stems cells are obtained using the methods of the invention in order to reverse the differentiation of a differentiated cell to provide a stem cell. In other cases the stem cells may originate from a different subject, but be chosen to be immunologically compatible with the intended recipient. In some cases the stem cells may be from a relation of the intended recipient such as a sibling, half-sibling, cousin, parent or child, and in particular from a sibling. The stem cells may be from an unrelated subject who has been tissue typed and found to have a immunological profile which will result in no immune response or only a low immune response from the intended recipient which is not detrimental to the subject. However, in many cases the stem cells, or the differentiated cells used to generate the stem cells, may be from an unrelated subject as the invention may be used to render the stem cell immunologically compatible with the intended recipient. For example, the stem cell and the recipient may or may not have a histocompatible haplotypes (e.g. HLA haplotypes).

In some cases the stem cells may be embryonic stem cells, foetal stem cells, neonatal stem cells, or juvenile stem cells. The embryonic, foetal, neonatal, or juvenile stem cells may be pluripotent stems cells and particularly totipotent stem cells. The cells may be from any stage or sub-stage of development, in particular they may be derived from the inner cell mass of a blastocyst (e.g. embryonic stem cells). The embryonic, foetal, neonatal or juvenile stem cells may be from, or derived from, any of the organisms mentioned herein. The embryonic, foetal, neonatal or juvenile stem cells may be human stem cells or non-human stem cells and in particular non-human animal stem cells (e.g. a non-human primate). The embryonic, foetal, neonatal or juvenile stem cells may be rodent stem cells and may in particular be mouse embryonic stem cells. In some cases the embryonic, foetal, neonatal or juvenile stem cells may be recovered and then used in the manufacture of medicaments to treat the same subject, typically at some stage in their life. In one embodiment, where embryonic, foetal, neonatal or juvenile stem cells are employed, they will be from already established foetal, embryonic, neonatal or juvenile stem cell lines. This will particularly be the case for human cells. In some cases the stem cells may be obtained from, or derived from, extra-embryonic tissues. The stem cells may be obtained from the umbilical cord and in particular from umbilical cord blood.

In certain jurisdictions, for reasons of public policy, the stem cells may not be totipotent stem cells that have the capacity to form a human being. This is particularly the case where the stem cells are human foetal or embryonic stem cells.

The invention is also applicable to stem cell lines. Stem cell lines are generally stem cell populations that have been isolated from an organism and maintained in culture. Thus the invention may be applied to stem cell lines including adult, foetal, embryonic, neonatal or juvenile stem cell lines. The stem cell lines may be a clonal stem cell line i.e. they may have originated from a single stem cell. In one preferred embodiment the invention may be applied to existing stem cell lines, articularly to existing embryonic and foetal stem cell lines. In other cases the invention may be applied to a newly established stem cell line.

The stem cells may be an existing stem cell line. Examples of existing stem cell lines which may be used in the invention include the human embryonic stem cell line provided by Geron and the neural stem cell line provided by Reneuron. In a preferred case the stem cell line may be one which is a freely available stem cell access to which is open and in particular such an existing stem cell line.

In the case of human embryonic stem cell lines, in a preferred case a pre-existing stem cell line will be used. In a particularly preferred embodiment of the invention, where a human embryonic stem cell line is used, the cell line may be one where the derivation process (which begins with the destruction of the embryo) was initiated prior to 9:00 p.m. EDT on Aug. 9, 2001. Preferably human embryonic stem cell lines may be ones created from embryos donated for reproductive purposes which were no longer needed for the original purpose, because, for example, they were surplus to requirements. Preferably informed consent will have been obtained for the use of the embryos to create the cell line. In a preferred case, the human embryonic stem cell line employed will meet the requirements announced by President Bush on 9 Aug. 2001 as being necessary for obtaining US federal funding for embryonic stem cell research. These include the stem cell lines recognised as meeting the requirements from BresaGen Inc. of Australia; CyThera Inc.; the Karolinska Institute of Stockholm, Sweden; Monash University of Melbourne, Australia; National Centre for Biological Sciences of Bangalore, India; Reliance Life Sciences of Mumbai, India; Technion-Israel Institute of Technology of Haifa, Israel; the University of California at San Francisco; Goteborg University of Goteborg, Sweden; and the Wisconsin Alumni Research Foundation.

Reference herein to stem cell generally includes the embodiment mentioned also being applicable to stem cell lines unless, for example, it is evident that the target cells are freshly isolated stem cells or the stem cells are resident stem cells in vivo. The invention is applicable to freshly isolated stem cells and also to cell populations comprising stem cells. The invention may also be used to control the differentiation of stem cells in vivo.

An initial step in the methods of the invention may be the isolation of suitable stem cells. Methods for isolating particular types of stem cells are well known in the art and may be used to obtain stem cells for use in the invention. The methods may, for example, be used to recover stem cells from the intended recipients of the medicaments of the invention. Cell surface markers characteristic of stem cells may be used to isolate the stem cells, for example, by cell sorting. Stem cells may be obtained from any of the types of subjects mentioned herein and in particular from those suffering from any of the disorders mentioned herein.

In some preferred embodiments stems cells may be obtained by using the methods of the invention to reverse the differentiation of differentiated cells to give stem cells. In particular, differentiated cells may be recovered from a subject, treated in vitro in order to produce stem cells, the stem cells obtained may then be manipulated as desired and differentiated before (and/or after) return to the subject. As stem cells typically represent a very small minority of the cells present in an individual such an approach may be preferable. It may also mean that stem cells are more easily derivable from specific individuals and may eliminate the need for embryonic stem cells. In addition, typically such an approach will be less labour intensive and expensive than methods for isolating the stem cells themselves. In some cases, the stem cells may be isolated from a subject, differentiated in vitro and then returned to the same subject. Such ex vivo methods are particularly preferred.

In some cases the target stem cells may be in situ, that is they may be present in a subject. Thus in a further aspect the invention provides for the use of a RNA in accordance with the invention which is capable of inducing differentiation of stem cells in the manufacture of a medicament for use in improving or rectifying tissue or cellular damage or a genetic disease. The invention also embraces methods that use isolated RNA in accordance with the invention, which is capable of inducing differentiation of stem cells in improving or rectifying tissue or cellular damage or treating a genetic disease. Such a method may, for example, be used for treating a degenerative brain disease or brain or spinal cord injury. It may also be used for the treatment of diseases such as liver disease, heart disease, skeletal or cardiac muscle disease and type I diabetes. Furthermore, it may be used to counteract age-related degenerative disease.

In such embodiments the stem cells may be any of the types of stem cells mentioned herein and may be in any of the organisms mentioned herein. The target stem cells may be present in any of the organs, tissues or cell populations of the body in which stem cells exist, including any of those mentioned herein. The target stem cells will typically be resident stem cells naturally occurring in the subject, but in some cases stem cells produced using the methods of the invention may be transferred into the subject and then induced to differentiate by transfer of RNA.

Various techniques for isolating, maintaining, expanding, characterising and manipulating stem cells in culture are known and may be employed. In some cases genetic modifications may be introduced into the genomes of the stem cells. Stem cells lend themselves to such manipulation as clonal lines can be established and readily screened using techniques such as PCR or Southern blotting. Techniques such as gene targeting or random integration may be used to introduce changes into the genome of the cells.

In some instances the stem cells may originate from an individual with a genetic defect. Modifications may then be made to correct or ameliorate the defect. For example, a functional copy of a missing or defective gene may be introduced into the genome of the cell. Gene targeting may be used to introduce desired specific changes and in particular to modify a defective gene to render it normal. Site-specific recombinases may be used to remove selective markers involved in the gene targeting. In a particular preferred embodiment, differentiated cells will be obtained from an individual with a genetic defect, stem cells obtained from the differentiated cells using the methods of the invention, the genetic defect corrected or ameliorated and then either the stem cells or differentiated cells obtained from them will be used for treating the original subject or in the manufacture of medicaments for treating the original subject.

In some cases the stem cells may be chosen because they have a specific genotype. For example the stem cells may be intended to produce differentiated cells to treat a subject with a genetic defect. The stem cells may lack the genetic defect. For example, the stem cells may be obtained from, or produced from differentiated cells obtained from, a relation of the subject who lacks the defect. For example, the cells may be derived from a sibling who does not have the disorder. In a preferred case the methods of the invention may be used to render the cells immunocompatible or more immunocompatible with the intended host.

RNA Molecules

In order to produce the desired changes in cell properties, the invention employs specific RNA.

The RNA employed is one that comprises RNA extractable from tissues or cells comprising the cell type or types that it is desired to induce the target cell to have a cell property of; or, for treatments of recipients, the RNA employed is one that comprises RNA extractable from tissue or cells comprising the tissue or cell type or types which it is desired to regenerate or repair. Thus, in the case where the aim is, for example, to induce differentiation of a stem cell into a desired differentiated cell type, the RNA provided to the target cell is typically an isolated RNA comprising a RNA sequence extractable from tissue or cells comprising the desired differentiated cell type or types. The isolated RNA may comprise a RNA extractable from or extracted from tissue or cells comprising the desired differentiated cell type or types.

The degree to which the source of the RNA is homogenous will be dictated in part by the specificity of the type of tissue that is desired. The RNA may be extracted from, or the RNA sequence may be derived from, a particular tissue type, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, skin, and gastrointestinal tract, lung, bone, cartilage, tendon, hair follicle, sense organ (for example, ear, eye), any gland either endocrine, exocrine, or paracrine, such as thyroid, thymus, pituitary, adrenal, pancreatic, reproductive system (for example, testicular, prostate, seminal vesicle, ovarian, uterine, fallopian, mammary), dental, vascular, digestive tract tissues (for example, stomach, gall bladder, intestines, colon). Such tissues are made up of a number of different cell types e.g. constituent cells of brain tissue include various sub-types of neurones and glial cells, vascular tissues, connective tissues and brain-resident stem cells. RNA may be from a specific type of tissue in a particular location, such as a left tibia or left frontal lobe Accordingly, a more homogeneous population of cells might include neurones and so where the desired cell fate is itself specific (for example, in the treatment of age-related brain disease), the RNA may be extracted from neurones, or the RNA sequence may be derived from neurones. More specifically again, the RNA may be from a specific neurone type such as cortical neurones. More specifically again, the RNA may be from a specific type of cortical neurones, such as dopaminergic cortical neurones. In embodiments such as these, the RNA is from a purified cell source.

In some embodiments, the RNA employed in the invention, derived from a particular tissue type or set of cells or cell lines or cell types, or a cell line or a single cell type, or the RNA sequence derived from such sources, may in addition use a source of such material which comes from a donor of a specific developmental stage. Accordingly the RNA may be derived from neurones from a particular developmental stage, where that developmental stage is the same as, or earlier than, or later than, the developmental stage of the intended recipient. For example, RNA used in the treatment of cardiac degeneration may be extracted from the cardiac tissue of a juvenile donor. Developmental stages include embryo, foetal, neonatal, juvenile, or adult, or any sub-stage of any of these stages.

In some embodiments the RNA employed in the invention, for the treatment of a tissue or organ in a recipient of a certain developmental stage, may be derived from a tissue or cell type or types that is related to that of the target tissue, but where the exact type of source tissue is only present at a different developmental stage. For example, dental tissue in an adult might be treated with RNA derived form the emergent dental tissue in a neonate or young juvenile.

Other preferred sources of homogenous, purified RNA for use in accordance with the present invention include pure preparations of foetal, neonatal or juvenile cells and pure preparations of embryonic stem cells.

In cases where it is desired to reverse the differentiation of a differentiated cell to a desired stem cell type, the RNA provided is typically an isolated RNA comprising RNA sequence extractable from the desired stem cell type or types which it is wished to obtain. The RNA may be extractable or extracted from cells comprising said desired cell type(s).

In some embodiments of the invention, for effecting regeneration or repair in vivo, the RNA employed is derived from stem cells, and is administered into the whole organism, or organ, or tissue. This can cause regeneration and activation of the recipient stem cell population per se, with a secondary consequential regenerative effect on the tissues normally supported by these stem cells. In this case, the RNA provided is typically isolated RNA comprising RNA sequence extractable from a stem cell type or types or stem cell active tissue(s). The RNA may be extractable or extracted from a stem cell type or types or stem cell active tissue(s). Examples of stem cell-rich tissues include foetal tissue and embryo tissue, or tissues from later developmental stages undergoing a phase of growth repair or regeneration.

Typically, a cellular RNA extract will comprise a heterogeneous population of species of different RNA molecules. Types of RNA molecules in a heterogeneous population can include messenger RNA (mRNA), ribosomal RNA (rRNA), transfer RNA (tRNA), heterogeneous nuclear RNA (hnRNA), small nuclear RNA (snRNA), small cytoplasmic RNA (scRNA), small nucleolar RNA (snoRNA), transcription-related RNAs, splicing-related RNAs, signal recognition particle RNAs, linear RNA, circular RNA, inhibitory RNA (e.g. siRNA), single-stranded RNA, double-stranded RNA, etc. In a preferred embodiment the RNA will comprise a RNA extract of tissues or cells comprising the desired cell type or types, in particular the RNA may comprise, or consist essentially of, an extract from the desired cell types.

Thus preferably a RNA rich extract is prepared from donor material. The donor material may, for example, be an organotypic source obtained post mortem. The donor material may also be obtained from the same source as the cells to be treated, or from the intended recipient of the cells to be treated. For example the RNA extract may be from an organ or tissue or cells isolated from an organ or a tissue. For example, the RNA extract may be from an organ, tissue or cells isolated from the group comprising, but not limited to, the brain, spine, heart, kidney, spleen, skin, the gastrointestinal tract or liver. In some embodiments, the source organ, tissue or cells may have been treated one or more times with the methods or medicaments of the present invention. The extract may be from a cell line of specific chosen phenotype, a primary cell culture, or a donor tissue of specific immunological profile.

Typically the RNA will comprise RNA sequence that is extractable from the same species as the target cell to be treated. Thus in cases where the target cell to which the RNA will be provided is an animal cell, the RNA will usually comprise a RNA sequence extractable from or a RNA extracted from an animal cell and in particular from the same species of animal as the target cell to be treated. Similarly, where the target cell is a plant cell, usually the RNA will comprise a RNA sequence extractable from or a RNA extracted from a plant cell and typically a plant cell of the same species as the target cell.

The RNA may comprise a RNA sequence extractable from or a RNA extracted from any of the organisms or groups of organisms mentioned herein. The RNA may comprise a RNA sequence extractable from or a RNA extracted from any of the stem cell types or differentiated cell types mentioned herein.

In some embodiments, the RNA may comprise a RNA sequence extractable from or a RNA extracted from a different developmental stage than the recipient of the cells to be treated. For example, the developmental stage may be more immature than that of the recipient of the cells to be treated. Alternatively, the developmental stage may be a more active cell generative stage. For example, the treatment of spinal cord lesions may be effected by treatment with RNA obtained from donor embryo tissue, sourced at neuralation. The developmental stage may also be one that shows increased stem cell activity. For example, in some preferred embodiments of the invention, the RNA may comprise a RNA sequence-extractable from or a RNA extracted from foetal, neonatal juvenile or embryonic developmental stages. For example, where the RNA is extractable from brain cells or tissue, the donor may be at a developmental stage when extensive neurogenesis is occurring, such as the foetal developmental stage. It has been demonstrated by the inventors that provision of RNA extractable from cells of an early developmental stage has advantageous effects, particularly in eliciting stem cell-mediated tissue repair.

The developmental stage may in alternative embodiments be less immature than that of the recipient of the cells to be treated or a less active cell generative stage. In some embodiments, the RNA may comprise a RNA sequence extractable from or a RNA extracted from a tissue that has been pre-treated (for example, chemically or physically) or pre-conditioned (for example, by exercise for muscle tissue or induction of a particular reproductive stage for reproductive tissue) in any way or ways which modify the activity of the extractable RNA. For example, the RNA may be extracted from tissue that has been stressed or damaged.

The alteration in a cell property using the RNA in accordance with the invention as discussed above may result in the target cell adopting an immunological profile similar to or the same as that of the organism from which the RNA is extractable from. The expression “immunological profile”, is intended to include the immunological properties of the target cell in the intended recipient. Thus the invention may be used to change the immunological profile of a target cell in a desired manner. This may be used to ensure that the cells produced, or products produced from them, have a specific immunological profile. In particular, the RNA provided to the target cells may therefore be chosen so that the resultant cells, or products from them, have an immunological profile so that they are not immunogenic in the intended recipient or produce a minor immune response which is not significant and that preferably does not result in a detrimental phenotype. Thus the RNA provided may in a preferred case be a RNA sequence extractable from or a RNA extracted from, and particularly a RNA extracted from, cells or tissues of the intended recipient or an immunologically compatible subject. Such methodologies will in particular be useful in the provision of allografts or xenografts to patients, to minimise or prevent the risk of rejection.

The ability to change the immunological profile of a cell may mean that the stem cells or differentiated cells to which the RNA is provided do not themselves have to necessarily be immunologically compatible with the intended recipient. This means that cells such as stem cells may not necessarily have to be isolated from the intended recipient and, for example, already existing stem cells or stem cells from a more convenient source may be used. It may also mean that cells and in particular stem cells with a specific desired genotype may be employed and converted to a compatible immunological profile. For example, the intended recipient may have a genetic defect, whereas the stem cells or differentiated cells to which the RNA is provided may be from a different subject that does not have the same defect. Using the invention the donor cells may be rendered immunologically compatible to the intended recipient and also compensate for the genetic defect.

The alteration in a cell property using RNA in accordance with the invention may therefore be used to change the immunological properties of cells, such that cells that are allogeneic or even xenogeneic with respect to the treated individual may be administered with a minimised risk of rejection of the cells. For example, pig cells treated with human RNA prior to injection may be introduced into human patients with a minimised risk of rejection, through alteration of the expression of cell surface molecules and their replacement with self molecules that would otherwise have been recognised as non-self by the treated individual. The isolated RNA may thus comprise a RNA sequence extractable from or a RNA extracted from a different species to that of the target cell to be treated.

The alteration in a cell property using the RNA in accordance with the invention as discussed above may be used to boost the immune function of a diseased patient. For example, a RNA sequence for use in treatment may be isolated from a patient or species that is immune or relatively immune to the disease, either through natural resistance or through vaccination. The RNA may have the effect of conferring resistance to the treated patient, for example, through inducing a desired immune function or property already possessed by the cells of the individual from which the RNA was extracted. One example is in the incidence of pathogenic or viral disease. In such cases, it may be that RNA extracted from immune cells, such as T cells, of a resistant individual of the same or different species confers the required immune function to the treated individual. An example might be the case of HIV, which has little adverse effect on chimpanzees or certain groups of humans. RNA extracted from immune cells of chimpanzees or these groups of humans might be administered to a human or to immune cells isolated from a human and then reintroduced, in order to confer resistance on the human patient to AIDS.

The alteration in a cell property using the RNA in accordance with the invention as discussed above may be used to reverse tumour growth. It is postulated herein that by exposing a tumour cell to a RNA sequence extractable from or a RNA extracted from a healthy cell, or a cell at an early developmental stage, the tumour cell may be induced to revert to a normal, healthy phenotype, or to become susceptible to elimination by the immune system or by genetic integrity maintenance systems for example, p53-mediated apoptosis.

Preparation of RNA

Various techniques exist for the extraction of donor RNA. Such techniques may be used to obtain the RNA to be provided to the target cells. Alternatively, such techniques may be used to provide RNA to identify the sequences of the necessary RNA molecules in the RNA extract (e.g. by fractionation and screening). Thus the invention includes a method of screening for a RNA sequence capable of conferring a desired property from one cell type to another, comprising the steps of:
i. extracting RNA from cells comprising a desired cell type;
ii. separating the extracted RNA into different fractions;
iii. providing a fraction to one or more test cells and/or test recipients;
iv. analysing the test cells or recipients for an altered property possessed by the desired cell type from which the RNA was extracted;
   wherein a fraction that confers the altered property onto a test cell or recipient is identified as comprising a RNA sequence capable of conferring the desired property.

This screening method identifies RNA sequences that are capable of conferring a desired property from one cell type to another by fractionating the RNA extract and analysing RNA function using an appropriate assay. One example of an appropriate assay is an experiment of the type described in Example 1 below. The assay comprises providing isolated RNA comprising RNA extractable from cells comprising particular cell type(s) to a population of cells; and determining whether a cell property is altered towards a property of said desired cell type(s). In this way, RNA in an extract can be identified as unnecessary for the purposes of the invention and can be omitted (e.g. to simplify or standardise a RNA composition), ultimately leaving a RNA molecule, or set of RNA molecules, which are responsible for the desired activity.

Accordingly, the present invention also envisages the use of specific RNA sequences, specific RNA subtypes, or particular RNA structures that have been identified as capable of conferring a desired property from one cell type to another in the RNA extract. Such RNA molecules may be synthesised artificially. In some cases, the RNA may be an artificial or synthetic RNA or a RNA analogue based on the sequence of the extractable sequences. The analogue may be one chosen for its stability or ability to enter the target cell or other desirable properties.

Accordingly, the RNA employed in the invention may also be one that comprises RNA sequence extractable from tissues or cells comprising the cell type or types that it is desired to induce the target cell to have a cell property of. Thus, in the case where the aim is, for example, to induce differentiation of a stem cell into a desired differentiated cell type, the RNA provided to the target cell may typically be an isolated RNA comprising RNA sequence extractable from tissue or cells comprising the desired differentiated cell type or types.

Alternatively, the RNA may, for example, be prepared from a donor source. Suitable techniques include preparation by either cold or hot phenol extraction methodologies. Alternatively, the RNA may be sourced from specific tissues or cells by employing commercially available kits and in particular those that are based on the denaturing of protein and separation of RNA via centrifugation. For example, in one preferred protocol (cold phenol) extraction, primary donor tissue or cells is/are homogenised in a volume of physiological saline. An equal volume of 95% saturated phenol is added and initially centrifuged at 18,000 rpm in an ultra-centrifuge for 30 minutes. The aqueous phase is retained and brought to a concentration of 0.1M MgCl₂ solution by the addition of 1M MgCl₂. Two volumes of ethanol are then added and this is allowed to precipitate for approximately 30 minutes. A final spin at 6,000 rpm for 15 minutes produces a RNA rich precipitate which can be retained and stored under ethanol. Alternatively, active RNA rich extracts may be prepared with any of the commercially available RNA extraction kits (such as, for example, RNAzol™). However, the precise methodology by which the RNA is extracted is generally not critical to the invention.

In some cases a specific fraction of a RNA extract may be employed. For example, the RNA population may be fractionated on the basis of size and a particular weight range of RNA species provided to the target cell. Fractionation may also be on the basis of weight, charge, or identifiable common chemical feature (for example, a structure, or the presence of a particular consensus or pattern of nucleotides) or any combination of size, weight or charge or common chemical feature.

In some embodiments the RNA employed may comprise or consist of mRNA sequences present in the extract. In some embodiments the RNA fraction or RNA molecule may be specific to affecting some parts of the differentiation process but not others. For example, one RNA type or molecule may only induce genetic change but have not other effects such as migration, terminal differentiation, integration or proliferation. In another embodiment, the RNA type or molecule may affect all factors other than genetic modification. In another embodiment, the RNA type or molecule may effect only the location of migration, or only the degree of proliferation, or only the phenotypic cell type of terminal differentiation, but not any other aspect. In some cases the RNA may comprise a mixture of sequences extractable from different cell types or tissues. For example, the RNA species may comprise a mixture of sequences extractable from two, three, four, five or more different cell types. In cases where it is desired to differentiate a stem cell, the RNA may, for example, be extractable from different cell types to produce a differentiated cell with characteristics of both cell types. In cases where the RNA is to be provided to a target cell that has a genetic defect, the RNA may be a mixture of sequences extractable from cells comprising and lacking the defect. For example, the RNA may comprise a blend of RNA extracts from cells from the subject with the defect and cells of the same type from another subject that lack the defect. In some cases specific sequences that are extractable from the desired cell type may not be present. For example, the transcript of a defective gene may be removed. The removal of specific sequences may, for example, be achieved, by selective degradation or by hybridisation. Ribozymes may be used to cleave specific sequences. RNase molecules may also be used with some degree of specificity.

Specific sequences may be added to or removed from the extractable sequences. For example, in some cases the RNA may originate from the subject intended to be the eventual recipient of the cells produced and the subject may lack a specific gene sequence or have a defective gene sequence. In such cases an additional RNA corresponding to a RNA encoding the expression product of the missing or defective gene may be added to the extract. In such cases, the targeted genetic sequences may be repaired, modified, removed or selectively degraded.

In cases where the RNA is one extractable from a stem cell, preferred stem cells include any of those mentioned herein and in particular adult stem cells. The stem cell may, for example, be a haematopoietic, bone marrow stromal or neuronal stem cell. In cases where the RNA is one extractable from a differentiated cell, the differentiated cell may be any differentiated cell and may be in particular an adult differentiated cell. In a preferred embodiment the differentiated cell may be selected from a bone marrow cell, a neuronal cell, or a haematopoietic cell. The differentiated cell may be from any mammalian organ for example such as the kidney, liver, heart, pancreas, central nervous system, reproductive organ or other organ.

In some embodiments, isolated RNA extractable from cells and used in the methods of the invention is natural in derivation. By this is meant that the RNA contains no non-natural sequences and entirely consists of RNA from the species to which the cell belongs. In some embodiments, the RNA contains no viral, exogenous retroviral or pathogen sequences. In some embodiments, the RNA is a homogenous mixture and contains no siRNA, mRNA or other types of interfering RNA. In some embodiments, the RNA may not encode protein (e.g. the RNA does not have in-frame start and stop codons flanking a protein-coding region). In some embodiments, the RNA is not extractable from neoplastic cells. In some embodiments, the RNA contains no double-stranded RNA of a kind that directly activates an anti-viral immune response (e.g. by binding to a Toll recpetor). In some embodiments, the RNA contains no antisense RNA (e.g. there is no RNA that is complimentary to the sense strand of a RNA transcript that is also present). RNA used according to the invention may be integrating or non-integrating. It may or may not be capable of replication. It may or may not have a 5′ cap. It may or may not have a poly-A tail. It may or may not act as a substrate for endogenous reverse transcriptase.

Modified RNA and Analogs

The invention generally involves the use of RNA. This RNA comprises a sequence that can be extracted from cells comprising a desired characteristic. Transfer of the RNA to a target cell causes desired changes in the target cell, with the changes being defined by the RNA.

As shown herein, the RNA in which the changes are defined is active even when delivered within a phenol extract of RNA from a starting cell. This phenol extract contains a variety of different RNA molecules. If the activity is associated with specific RNA molecules and/or sequences within the extract then, to simplify preparation and quality control, it is preferred to deliver just the specific RNA rather than a complex mixture. The specific RNA can be prepared by purification from the RNA extract, or can instead be prepared synthetically or artificially (e.g. by chemical synthesis, at least in part, or by purification after transcription of the specific RNA from a template nucleic acid).

Thus the invention provides a process for preparing a RNA for use with the invention, comprising the step of synthesising the RNA by chemical means, at least in part. The invention also provides a process for preparing a RNA for use with the invention, comprising the steps of: contacting a template for said RNA with a RNA polymerase, whereby the polymerase can interact with the template to produce said RNA. The RNA polymerase could be a RNA-dependent RNA polymerase, but will typically be a DNA-dependent RNA polymerase (i.e. the template is preferably DNA, e.g. in the form of a plasmid).

The RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases. Bases such as pseudo-uridine, methyl-cytosine, and inosine may be present in such RNA molecules. It is also possible to include DNA nucleotides to form a DNA/RNA chimera. The use of modified backbones is a preferred feature of modified RNA molecules of the invention.

RNA analogs and mimics can also be used. Polymers that mimic natural RNA structures can be prepared and used with the invention etc. e.g. as described by Kirshenbaum et al. (1999). These modified molecules and analogs can be considered as “RNA” herein even if, from a strict chemical viewpoint, they are not simply ribonucleic acid.

Provision of RNA to Target Cells

The RNA may be provided to the target cells in vitro or in vivo. The RNA may also be used in the manufacture of medicaments for the provision of the RNA to the target cells in situ. This is particularly the case where the RNA is provided to target cells in the animal body. In the case of plants the invention also provides methods for providing the RNA to the target cells both in vitro and in vivo. The RNA may be provided to the target cells by any suitable technique.

A number of methods for the provision of nucleic acid molecules to cells are known and these may be employed. For example, suitable techniques may include calcium phosphate transfection, DEAE-Dextran, electroporation, liposome encapsulation, liposome-mediated transfection, microsphere encapsulation, transduction using viral envelope particles and microinjection. The calcium phosphate precipitation method of Graham & van der Eb (1978) may be employed. General aspects of mammalian cell host system transformations have been described in U.S. Pat. No. 4,399,216 and may be employed. For various techniques for transforming mammalian cells, see Keown et al. (1990) and Mansour et al. (1988). In some cases the RNA or the enclosed RNA may be bound to chemical agents that enhance uptake by the target cells. For example the RNA of RNA-containing particles may be linked to an antibody specific to an appropriate receptor. Such a targeting chemical may increase uptake by all cell types, or may have an effect which is specific to a particular cell type or stem cell type. As an alternative, RNA can be administered without being bound to such reagents e.g. naked RNA. In some cases the RNA may simply be added to the culture medium of the cells for a suitable period of time. For example, the cells and RNA may be cultured together for from 1 minute to 10 days, preferably from 1 hour to 5 days, more preferably from 6 hours to 2 days. In a preferred embodiment the RNA may be cultured with the cells for 12 or 24 hours and in particular for 12 hours. Similar time periods may be employed where the RNA is provided in the form of liposomes comprising the RNA sequences.

In other embodiments the RNA may be used in treatment methods or in the manufacture of medicaments which will allow in vivo provision of the RNA to stem cells or to other cells. In such cases the RNA is typically formulated so that the medicament is in a suitable form for administration to the intended subject.

The medicament may be in a form where the RNA is in liposomes to facilitate delivery or alternatively encapsulated within viral envelope particles. The RNA may be present as naked RNA molecules or RNA molecules complexed with proteins and in particular proteins known to increase uptake of nucleic acids into cells.

The medicament may be administered in conjunction with other treatments given prior, simultaneously or subsequently which increase the time for which the medicament remains in an active state, in vitro or in vivo. For example the use of a known RNase inhibitor could be used for such treatment. Alternatively saturating dose of inactive or sacrificial RNA may be given to block the existing RNase activity.

The medicament may be administered in conjunction with other treatments given systemically or locally, prior, simultaneously or subsequently which increase the uptake or effect of the medicament in vitro or in vivo. For example molecules secreted in a local or systemic manner following tissue damage may enhance uptake of the medicament. Such molecules may originate from the damaged tissue per se, or from a stem cell source. In another example, known non-RNA inducers of tissue differentiation of specific tissues culture may be used in conjunction with the RNA of this method in vitro for example, the use of retinoic acid to aid differentiation of neuronal tissues. In another example known non-RNA supports of tissue culture may be used in conjunction with the medicament, for example, basic fibroblast growth factor in the culture of spinal neurons.

The medicament comprising the RNA may be delivered by any suitable route. For example, the medicament may be administered parenterally and may be delivered by an intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, transdermal, intranasal (or other mucosal), pulmonary, inhalation, or other appropriate administration route. The medicament may be administered directly to the desired organ or tissue or may be administered systemically. In particular preferred routes of administration include via direct organ injection, vascular access, or via intra-muscular, intravenous, or subcutaneous routes. The RNA may be formulated in such a way as to facilitate delivery to the target cells.

The RNA may be provided on metallic particles. In some cases the medicament may be intended to be administered so that naked RNA is provided to the target cells. In cases where the RNA is provided present in liposomes or other particles, there may be targeting molecules present on the surface of the particles to allow targeting to the intended stem cells. For example, the particles may comprise ligands for receptors on the target stem cells or target differentiated cells. In one preferred embodiment, RNA is delivered to the cells via liposomes prepared after the methodology of Felgner et al. (1987) Other suitable liposomes include immunoliposomes (e.g. U.S. Pat. No. 4,957,735).

RNA preparations may also be administered to an organism with cells, such as stem cells. Administration may be simultaneous, separate or sequential. Cells and RNA of the invention may also be administered in simultaneous, separate or sequential application with other therapies effective in treating a particular disease. In one embodiment, RNA extractable from one or more stem cell types or stem cell active tissue(s) may be administered in simultaneous, separate or sequential application with cells, such as stem cells. For example, in preferred embodiments, whole embryo RNA, foetal RNA, neonatal RNA or juvenile RNA is administered in simultaneous, separate or sequential application with stem cells, particularly bone marrow stem cells. It is shown here that stem cell mediated tissue repair and regeneration is improved by co-injecting embryo-derived RNA fractions with stem cells.

Cells and Pharmaceutical Compositions

The invention provides cells obtained by the methods of the invention. The cells may be provided as frozen cells in a suitable receptacle. The cells may be provided in culture. Extracts of the cells are also provided such as whole cell extracts.

The invention also provides pharmaceutical compositions comprising the various RNA molecules, stem cells, and/or differentiated cells of the invention. The RNA molecules, stem cells and differentiated cells may be formulated with standard pharmaceutically acceptable carriers and/or excipients as is routine in the pharmaceutical art. Techniques for formulating cells and nucleic acids may be employed as appropriate. The cells or RNA may be provided in physiological saline or water for injections. The exact nature of a formulation will depend upon several factors including the particular substance to be administered and the desired route of administration. Suitable types of formulation are fully described in Remington's Pharmaceutical Sciences, 19^{th} Edition, Mack Publishing Company, Eastern Pennsylvania, USA, the disclosure of which is included herein of its entirety by way of reference. RNA-based pharmaceuticals are known in the art. For example, ‘Ampligen’ (Hemispherx Pharma) is a medicament comprising double-stranded RNA molecules.

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sugars, and lipid aggregates (such as oil droplets or liposomes). Such carriers are well known to those of ordinary skill in the art. Compositions may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in Gennaro (2000).

Compositions of the invention will generally be in aqueous form (e.g. solutions or suspensions), but they may alternatively be in fried form (e.g. lyophilised). Liquid formulation allows the compositions to be administered direct from their packaged form, without the need for reconstitution in an aqueous medium, and are thus ideal for injection. Compositions may be presented in vials, or they may be presented in ready-filled syringes. The syringes may be supplied with or without needles. A syringe will include a single dose of the composition, whereas a vial may include a single dose or multiple doses.

Compositions of the invention may be packaged in unit dose form or in multiple dose form. For multiple dose forms, vials are preferred to pre-filled syringes. Effective dosage volumes can be routinely established, but a typical human dose for injection has a volume of 0.5 ml.

The pH of the composition for patient administration is preferably between 6 and 8, preferably about 7. Stable pH may be maintained by the inclusion of a buffer in the composition (e.g. a histidine or phosphate buffer). The composition will generally be sterile and/or pyrogen-free. Compositions may be isotonic with respect to humans. Compositions of the invention may include sodium salts (e.g. sodium chloride) to give tonicity.

Compositions of the invention may include an antimicrobial, particularly when packaged in multiple dose format. The various RNA preparations and compositions used to provide the USA discussed herein to the target cell may also comprise agents to increase the stability of the RNA. For example, they may comprise RNase inhibitors or other agents that stabilise and/or protect the RNA from degradation. The RNA preparations may also have been treated to remove other kinds of molecules, for example protease or DNase treatment may have been used to remove protein and/or DNA. Thus the composition may be substantially free from DNA and/or protein.

Some pharmaceutical compositions of the invention include combinations of RNA extracted from cells or tissues according to any one of the embodiments described above, either alone or in combination with stem cells. The cells of the invention may be administered to a patient together with other active agents, such as one or more anti-inflammatory agent(s), anti-coagulant(s) and/or human serum albumin (preferably recombinant), typically in the same injection. The cells will generally be administered to a patient essentially in the form in which they exit culture. In some cases, however, the cells may be treated between production and administration. The cells may be preserved (e.g. cryopreserved) between production and administration. Cells may be present in a maintenance medium.

Specific combinations of particular interest include RNA extracted from brain tissue, neurone cells, cortical neurones and the like, with stem cells, for example bone marrow mesenchymal stem cells; spine RNA with stem cells, for example with bone marrow mesenchymal stem cells; foetal RNA with stem cells, for example with bone marrow mesenchymal stem cells; embryo-derived RNA, such as embryonic stem cell RNA with stem cells, for example with bone marrow mesenchymal stem cells. Examples of treatments would include: for Alzheimer's Disease treatment of bone marrow stem cells with foetal brain RNA; for treatment of Parkinson's Disease, bone marrow stem cells with RNA from a culture of dopaminergic neuronal cells obtained form a juvenile donor; for heart disease, bone marrow stem cells treated with RNA from a juvenile or adult cadaver; for diabetes CD34+ circulation stem cells treated with RNA from pancreatic islet cells form the cadaver of a normal adult. For multiple sclerosis, bone marrow stem cells treated with RNA derived from primary cultures of oligodendroglia. Such compositions are for simultaneous, separate or sequential administration to a patient suffering from a disease that is amenable to treatment according to the invention (although in each case treatment may also be effected by direct administration of only the RNA to the recipient). Examples of such diseases are presented above. Where stem cells and RNA are to be administered together, they may be packaged separately or in admixture, and they may then be administered separately or in admixture.

A therapeutically effective amount of the medicament, compositions, cells or RNA molecules will be administered to a subject. The dose may be determined according to various parameters, especially according to the substance used; the species, age, weight and condition, including immuno-status, of the patient to be treated; the route of administration; and the required regimen. A physician will be able to determine the required route of administration and dosage for any particular patient. The dose may be determined taking into account the age, weight and conditions of the subject to be treated, the type and severity of the degeneration and the frequency and route of administration.

The amount of RNA provided to the target cells will be sufficient to bring about the necessary desired alteration in a cell property. For example the concentration of RNA (e.g. in a composition of the invention) may be from long to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. These concentrations may apply to in vitro or in vivo applications. In some cases, a total of 100 ng to 0.1 g, preferably from 1 μg to 50 mg, more preferably from 110 μg to −10 mg, still more preferably from 250 μg to 1 mg of RNA may be administered. Any suitable concentration and/or amount of RNA may be provided. A wide range of concentrations and/or amounts of RNA may be employed and the precise concentration and/or amount may be varied according to the method of delivery of the RNA to the target cells or tissues, the source of the RNA and whether the RNA is provided in vitro or in vivo. It is routine to optimise the amount of RNA provided to the target cells in order to bring about the desired alteration.

The invention provides a pharmaceutical composition comprising a RNA of the invention (including RNA mimics, analogs, and modified RNAs), wherein the composition: (i) has a pH between 6 and 8; (ii) includes a buffer; (iii) is sterile; and (iv) is substantially pyrogen-free. The RNA in the composition is preferably homogenous. The RNA is preferably the active pharmacological agent within the composition. The composition is preferably located within a container that is labelled to indicate the composition's pharmaceutical purpose.

Medicaments and Methods for Treating Subjects

The stem cells, RNA and differentiated cells provided by the invention may be used to treat a number of disorders, and in the manufacture of appropriate medicaments. In particular, the RNA and cells of the invention may be used in improving or rectifying tissue or cellular damage or genetic disease, and in the manufacture of appropriate medicaments.

The invention may employ a number of approaches to treat such disorders and to provide appropriate medicaments. In particular, administration of the medicaments of the invention to a subject to be treated may result in:
(a) administration of a RNA of the invention to a subject in order to induce differentiation of cells, such as stem cells, in situ; or administration of a RNA of the invention to a subject in order to induce mobilisation, migration, integration, proliferation and differentiation of cells, such as stem cells, in situ;
(b) administration of stem cells obtained by the invention to a subject;
(c) administration of differentiated cells obtained using the methods of the invention to a subject;
(d) administration of a RNA of the invention to the subject prior to, in conjunction with or after administration of cells (e.g. stem cells or differentiated cells), which cells may or may not have been altered according by the methods of the invention; and/or
(e) treatment of stem cells with a RNA of the invention prior to, or after, administration of the cells to a subject.
(f) administration of cells or stem cells with altered properties obtained using the methods of the invention to a subject;
   Generally, in the aspects of the invention under b) to f), in some cases it may be desired to use the methods of the invention to provide a cell type which is missing, depleted in number or functionally defective. The cells of the invention may be provided to a specific site or to a larger region. For example, the cells may be provided to a site of tissue or organ damage or injury such as a wound or broken bone. The cells may be provided to the site of a nerve injury and in particular to a spinal column injury. The cells may be provided to a damaged or diseased liver, kidney, heart or other organ. In the case of damaged or defective cardiac muscle disease such as in heart disease, dead or damaged cells can be augmented or replaced. Similarly cells can be provided to subjects with liver disease such as liver fibrosis, or other types of liver damage. Typically differentiated cells, or cells with altered properties (latent or evident) obtained using the methods of the invention will be provided, in some cases however stem cells obtained using the methods of the invention may be provided and allowed to differentiate in situ.
   a) RNA Therapy

It is shown herein that administration of RNA extracted from brain cells to a subject has the effect of stimulating resident stem cells in a patient to thicken brain cortex. Furthermore, RNA prepared from developmental stages known to show increased stem cell activity has been demonstrated to stimulate endogenous repair mechanisms. In one embodiment of methodology (a) above, the administration of a RNA in accordance with the invention to the subject induces differentiation of cells, such as stem cells, in situ in such a way as to promote stem cell-mediated functional repair. The administration may induce mobilisation, migration, integration, proliferation and differentiation of the cells in situ so as to promote stem cell-mediated functional repair.

Accordingly, this aspect of the invention provides an in vivo method of directing differentiation of cell fate towards a function or property of one or more desired cell types or tissues comprising providing isolated RNA comprising a RNA sequence extractable from cells comprising said desired cell type(s) to a population of cells under conditions whereby the desired differentiation of said cells is achieved. The RNA may be extractable or extracted from cells comprising said desired cell type(s). The population of cells is preferably a tissue, such as an isolated tissue grown outside the body, or an organism such as a human patient.

The invention also provides a method of improving or rectifying tissue or cellular damage or a genetic disease in a subject, the method comprising inducing totipotent or pluripotent resident stem cells in the subject to differentiate (e.g. with mobilisation, migration, integration and proliferation) into one or more desired cell types (e.g. at the target location), which method comprises providing isolated RNA sequence comprising RNA extractable from tissue or cells comprising said desired cell type(s) to the resident stem cells in situ, whereby the desired differentiation (e.g. with mobilisation, migration, integration and proliferation) of said stem cells is achieved. The RNA may be extractable or extracted from cells comprising said desired cell type(s). The method of the invention may be used to improve stem cell-mediated repair, either in vivo or in vitro.

In one embodiment, this aspect of the present invention provides a method of inducing totipotent or pluripotent stem cells in tissue of an animal or plant to differentiate into one or more desired cell types, which comprises providing isolated RNA comprising RNA sequence extractable from tissue or cells comprising said desired cell type(s) to said stem cells under conditions whereby the desired differentiation of said stem cells is achieved. The RNA may be extractable or extracted from cells comprising said desired cell type(s). The stem cells reside and are exposed to the RNA in situ, in the organism.

The invention may be used to treat, ameliorate and reverse tumour growth. It is postulated above that by exposing a tumour cell to RNA sequence extractable from a healthy cell, or a cell at an early developmental stage (such as foetal RNA, embryonic cell RNA, neonatal RNA or juvenile RNA), the tumour cell may be induced to revert to a more normal, healthy phenotype. In this aspect of the invention, the RNA sequence for treatment of tumour cells may be derived from healthy cells isolated from the patient or a related individual, an unrelated individual, or even a different species. Preferably, the RNA is from a closely related individual. The cell type from which the RNA for treatment is derived is preferably a similar cell type or the same cell type as the tumourigenic tissue. Numerous techniques exist for the typing of tumour cells, as the skilled reader will be aware.

In other embodiments of the invention, the method may be used to confer desired properties of one cell type onto another, optionally in situ in a patient. For example, in the same way that a desired immunological profile may be conferred onto target cells, desired properties possessed by a particular cell type may be conferred on target cells by extracting RNA from the cell type with the desired properties and exposing target cells to this RNA. Examples include extraction of RNA from muscle cells of trained athletes, so as to confer a desired function in a treated patient; transferral of resistance to disease from a vaccinated or naturally resistant individual; and boosting the immune function of a diseased patient.

In some cases the medicaments and methods of the invention may involve the RNAs of the invention being provided to the target stem cells in situ. This may result in resident stem cells differentiating to give rise to the desired differentiated cell type. Such an approach may be used for any of the above-mentioned conditions and disorders. In such an approach the RNA will typically be delivered so that it only affects a relatively localised population of stem cells. Preferably, the stem cells targeted may be those that give rise to the particular cell type involved in the disorder, but this may not always be the case. For example, the subject may have an immune system disorder and haematopoietic stem cells may be targeted.

Delivery to the chosen population of stem cells may be achieved by providing the RNA locally, such as to the appropriate tissue or organ. For example, the administration of the RNA may be intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, or inhalation to a site of interest. The RNA may, for example, be provided by local injection. The RNA may be provided by injection into a blood vessel or other vessel that leads to the desired target site. The RNA may be administered by local injection to the desired tissue. The RNA may be administered by any of the routes mentioned herein such as intra-muscular injection or by ballistic delivery. In some cases the localised delivery may be achieved because the RNA is provided in a form that specifically targets the RNA to the chosen cells. For example, the RNA may be provided in liposomes or other particles that have targeting molecules for the specific desired stem cell type. In preferred embodiments the RNA may be administered via direct organ injection, vascular access, or via intramuscular, intra-peritoneal, or sub-cutaneous routes.

In one preferred embodiment administration of a RNA is achieved as follows:
a RNA extract is prepared from desired tissue type including any of those mentioned herein;
the RNA is injected either directly to affected organ or via systemic delivery as defined above; and
the RNA induces resident stem cell differentiation resulting in, for example, proliferation of the desired cell type, migration and repair.

In some embodiments, the RNA sequence is extractable from or extracted from one or more differentiated cell types. For example, in a specific embodiment, the RNA is derived from primary tissue, such as brain tissue. In other embodiments, the RNA is extractable from one or more stem cell types or stem cell active tissue(s). For example, in a specific embodiment, the RNA is derived from adult stem cells, such as bone marrow stem cells. In another specific embodiment, the RNA is derived from foetal, neonatal, juvenile or embryonic tissue(s).

b) Therapy Using Stem Cells

In some cases, stem cells obtained using the methods of the invention may be administered to the subject, rather than differentiated cells or stem cells with properties altered by the method by RNA from cells other than stem cells. The stem cells may be administered to augment those already present in the subject. In some cases the stem cells may be administered to a site of tissue damage and then allowed to differentiate naturally. In some case stem cells may be added to augment those already present as the additional stem cells lack some defect present in the resident stem cell population and in particular a genetic defect. For example, the subject may have a genetic disorder that results in the absence of a particular cell type or cell lineage, a reduction in number of a particular cell type or cell lineage or in a particular cell type or lineage being defective. Stem cells lacking the defect may then be transferred to compensate for the genetic defect as they can give rise to the desired cell type or lineage or so that the cells or lineages they give rise to lack the functional defect. The stem cells administered may proliferate to maintain their number and also give rise to differentiated cells and hence have a long lasting effect reducing the need for frequent treatment. Indeed the transfer of the stem cells may result in a permanent cure or amelioration of the condition.

A subject may, for example, have an immunodeficiency caused by a genetic defect. Transferring a population of stem cells obtained using the invention that do not have the defect may be enough to treat the disorder as a proportion of the immune cells generated will lack the defect and be functional. In some cases the disorder may result from an in infection and in particular a viral infection and the stem cells may have some modification that prevents the cells becoming infected. In other cases stem cells obtained using the methods of the invention may be transferred to subjects whose own stem cell population has been depleted. For example, the subject may have been exposed to radiation or chemical agents that result in a decrease in stem cell number.

In a preferred embodiment of the invention, in cases where stem cells are transferred to a subject they will be derived from the same subject using the invention to produce stem cells from their differentiated cells. In other cases, the stem cells may be differentiated from an immunologically compatible unrelated individual. In some cases, the differentiated cells used to obtain the stem cells may be from a different individual, but the RNA provided to the cells may be from the intended recipient or a genetically compatible recipient. The provision of the RNA may result in the stem cells being immunologically compatible to the intended recipient.

c) Therapy Using Differentiated Cells

In a further embodiment differentiated cells obtained using the invention may be administered to the subject. In a preferred embodiment, the stem cells used to obtain the differentiated cells may have been obtained or derived from the intended recipient. Any of the differentiated cell types mentioned herein may be administered and the subject may be suffering from any of the disorders and conditions mentioned herein.

The differentiated cells may be administered to the localised site affected by the disorder. For example, they may be delivered to the pancreas in the case of diabetes, to the spinal nerve in the case of spinal injury, to the brain for brain disorders and so on. In some cases the differentiated cells may be provided to the subject present on, or as part of, a structure. For example, stents coated with ifferentiated cells may be inserted into a blood vessel or liver cells may be provided on a matrix to damaged or diseased liver.

The invention also provides a method of improving or rectifying tissue or cellular damage or a genetic disease in a subject, the method comprising administering to the subject an effective amount of differentiated cells obtained in vitro by inducing totipotent or pluripotent stem cells of a stem cell line or obtained from a tissue of an animal to differentiate into one or more desired cell type(s), which comprises providing isolated RNA comprising RNA extractable from tissue or cells comprising said desired cell type(s) to a cell culture of said stem cells under conditions whereby the desired differentiation of said stem cells is achieved. In a particularly preferred method the stem cells used are obtained from the subject to be treated. In an even more preferred embodiment, the stem cells used are obtained by using the methods of the invention to induce the reversal of differentiation of differentiated cells in vitro to provide the stem cells and in particular the differentiated cells used to obtain the stem cells are obtained from the subject.

d) RNA and Cell Combination Therapy

RNA according to the invention may be applied to a cell population in conjunction with other active agents, including, for example, stem cells (with or without altered properties, latent or evident) or differentiated cells. The RNA and other active agents may be administered simultaneously, sequentially or separately.

Combinations of these integers may also be employed. For example, medicaments comprising the stem cells may be administered to the subject and then a medicament comprising a RNA capable of inducing differentiation in accordance with the invention may be administered in order to induce their differentiation or alteration in situ In an alternative methodology, the stem cells may be introduced subsequent to the introduction of the RNA. Cells and RNA of the invention may be administered in simultaneous, separate or sequential application. Cells and RNA of the invention may also be administered in simultaneous, separate or sequential application with other therapies effective in treating a particular disease. In one embodiment, RNA extractable from one or more stem cell types or stem cell active tissue(s) may be administered in simultaneous, separate or sequential application with cells, such as stem cells. For example, in preferred embodiments, whole embryo RNA, foetal RNA, neonatal or juvenile RNA is administered in simultaneous, separate or sequential application with stem cells, particularly bone marrow stem cells. It is shown here that stem cell mediated tissue repair and regeneration is improved by co-injecting embryo-derived RNA fractions with stem cells.

e) RNA Treatment of Cells Prior to Administration

Administration of the medicaments of the invention according to this aspect of the invention may involve treatment of stem cells with a RNA according to the invention prior to administration of the stem cells to a subject. This approach has the effect of enhancing the mobilisation, migration, integration, proliferation and/or differentiation of the stem cells in the subject. In preferred embodiments, the stem cells are treated with RNA sequence that is extractable from or extracted from one or more differentiated cell types, in accordance with any one of the embodiments of the invention described above. For example, in one specific embodiment, bone marrow stem cells may be pre-treated with brain RNA prior to their administration to a subject, such as a subject suffering from age-related damage to the brain. This has been demonstrated herein successfully to reverse and thus treat age-related disease of the brain. In another specific embodiment, bone marrow stem cells are pre-treated with spine RNA, prior to their administration to a subject, such as a subject suffering from motor neurone disease. This has been demonstrated herein to be effective in an acknowledged model of motor neurone disease.

f) Therapy Using Stein Cells with Altered Properties

In a further embodiment stem cells with altered properties, obtained using the invention, may be administered to the subject. In one preferred embodiment, the cells are administered relatively soon after treatment in vitro with RNA, at a time when several of the altered properties are latent rather than evident, and where the later stages of migration, integration, proliferation, and differentiation may occur in vivo in the recipient. In another embodiment, the cells are administered when proliferation and differentiation have been evidenced. In a preferred embodiment, the stem cells used to obtain the cells with altered properties, evident or latent, may have been obtained or derived from the intended recipient. Cells with altered properties, latent or evident, related to any of the differentiated cell types mentioned herein may be administered and the subject may be suffering from any of the disorders and conditions mentioned herein.

The cells with altered properties, latent or evident, may be administered to the localised site affected by the disorder. For example, they may be delivered to the pancreas in the case of diabetes, to the spinal nerve in the case of spinal injury, to the brain for brain disorders and so on. In some cases the said cells may be provided to the subject present on, or as part of, a structure. For example, stents coated with said cells may be inserted into a blood vessel or liver cells may be provided on a matrix to a damaged or diseased liver. In another embodiment the cells with altered properties may be administered on a more general basis, for example by into the circulation, peritoneum, into the cerebrospinal fluid, intrapleurally.

Delivery of the cells with altered properties, latent or evident, may be achieved by providing the cells locally, such as to the appropriate tissue or organ. For example, the administration of the cells may be intravenous, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, inhalation, to a site of interest. The cells may, for example, be provided by local injection. The cells may be provided by injection into a blood vessel or other vessel that leads to the desired target site. The cells may be administered by local injection to the desired tissue. The cells may be administered by any of the routes mentioned herein such as intra-muscular injection. In preferred embodiments the cells may be administered via direct organ injection, vascular access, or via intramuscular, intra-peritoneal, or sub-cutaneous routes.

The invention also provides a method of improving or rectifying tissue or cellular damage or a genetic disease in a subject, the method comprising administering to the subject an effective amount of cells with altered properties, evident or latent, obtained in vitro by altering the properties of totipotent or pluripotent stem cells of a stem cell line or obtained from a tissue of an animal to, which comprises providing isolated RNA comprising RNA extractable from tissue or cells comprising said desired cell type(s) or tissue to a cell culture of said stem cells under conditions whereby the desired alteration of properties of said stem cells is achieved. In a particularly preferred method the stem cells used are obtained from the subject to be treated. In another preferred embodiment, the stem cells used are obtained by using the methods of the invention to induce the reversal of differentiation of differentiated cells in vitro to provide the stem cells, and in particular the differentiated cells used to obtain the stem cells are obtained from the subject.

The invention may be used to treat or ameliorate degenerative brain disease, brain or spinal cord injury or other neuronal disorders. In preferred embodiments the cells may be provided to a subject suffering from a degenerative disease and in particular an age related degenerative disease. The disease or damage to be treated with the medicaments of the invention may affect the brain. The subject may, for example, be suffering from a degenerative brain disease. Examples of brain disorders include, in particular, Parkinson's disease, Parkinsonian type disorders, Alzheimer's, dementia, other age related brain pathologies and Motor neurone disease. Multiple sclerosis may also be treated. Another disorder that may be treated is diabetes and particularly type 1 and type 2 diabetes, by providing insulin producing islet of Langerhans cells to replace or augment the defective cells. The invention may also be used for subjects suffering from disorders caused by damage to joints such as, for example, arthritis.

The invention also provides an agent for improving or rectifying tissue or cellular damage or a genetic disease, the agent comprising the RNA or differentiated cells (or cells with altered properties, latent or evident) as defined herein, or a combination of both. For example the invention provides for the treatment of degenerative diseases and age-related degeneration of any organ for example, heart disease, congestive heart failure, cardiac valve dysfunction, venous valve dysfunction, degenerative kidney disease, and degenerative liver disease. The invention also provides for regeneration of tissue after damage due vascular accident for example, ischemia, thrombosis, aneurism, and pressure sores.

The invention also provides a method for regeneration, repair or replacement of tissue(s) damaged or lost through pathology, age, or trauma of any description. For example the invention provides a method for regeneration and repair following traumatic damage to the spinal column.

In the above methods of treating a subject the stem cell, differentiated cells, altered cells, RNA, method of providing the RNA and other aspects may be as defined anywhere herein. In respect of the above agents, the RNA or differentiated cell or altered cell may be any defined herein.

In Vitro Methods for Reversing the Differentiation Of Differentiated Cells in Order to Provide Stem Cells

The invention provides methods for reversing the differentiation of differentiated cells to produce stem cells. The invention thus provides a method of reversing in vitro the differentiation of differentiated cells of a cell line or obtained from the tissue of an animal or a plant to produce a desired type or types of totipotent or pluripotent stem cell(s) or stem cell line(s), which comprises providing isolated RNA comprising RNA sequence extractable from the desired type(s) of stem cell or stem cell line to a cell culture of said differentiated cells whereby the desired reversal of differentiation of the differentiated cells into said type(s) of stem cell or stem cell line type(s) is achieved. The RNA may be extractable or extracted from cells comprising said desired cell type(s).

As existing methods for isolating stem cells are often laborious and require large amounts of material from a subject, the ability to reverse the differentiation of differentiated cells to provide stem cells provides a more convenient alternative which is less time consuming, more economical and less invasive. In particular, where it is desired to obtain stem cells from a subject suffering from a disorder it simply may not be practical to isolate stem cells directly from such a subject due to the invasive nature of the procedure needed to recover stem cells or the limited amount of material recoverable from the patient. The method of the invention also has the advantage that a wide range of stem cells can be obtained and that the stem cells obtained have the capacity to differentiate into a wide range of differentiated cell types.

The differentiated cells employed in the method may be any suitable differentiated cells including any mentioned herein. In particular, the differentiated cells may be cells that are readily accessible. The differentiated cells may be obtained from skin samples or from the buccal cavity. In a particularly preferred case the differentiated cells may be fibroblasts and particularly skin fibroblasts. In some cases the cells may be obtained from a bodily fluid and in particular from blood. In some cases white blood cells may be used such as, for example, lymphocytes.

The RNA may be provided using any of the methods described herein. After provision of the RNA to the cells the resulting stem cells may be cultured and passaged. The reversal of differentiation may be confirmed by examining cell morphology and by checking for the presence of stem cell specific markers. The ability of the stem cells for self-renewal may also be confirmed with the cells being passed through several passages to check that no differentiation occurs. The ability of the cells to differentiate into specific cells may also be examined. The karyotype of the obtained stem cells may be determined and in particular it may be checked to ensure that the karyotype of the cells is stable over several generations. The stem cells may be expanded. Samples of the stem cells may be frozen for later use or reference. In particular, samples of cells that have undergone low numbers of passages may be frozen, such as cells that have undergone ten or less, five or less, two or one passage(s). Clonal stem cell lines may be established from the general stem cell population and selected for specific desired characteristics such as their developmental capacity.

The resultant stem cells may also be manipulated to introduce desired genetic modifications. For example, if the original differentiated cells comprised a genetic defect the defect may be corrected. Sequences that can functionally compensate for missing or defective sequences may be introduced. Functional copies of missing or defective genes or other sequences may be introduced. Techniques such as PCR and Southern blotting may be used to screen for and identify clones with the desired modifications. The obtained stem cells may be differentiated and then assessed to check that the defect has been corrected. Techniques such as gene targeting may be used to introduce site-specific changes to the endogenous copies of genes. These may be employed in conjunction with site-specific recombinases to remove selectable markers used in the targeting. In particular, single gene disorders may be corrected using such techniques. Both dominant and recessive disorders may be corrected.

The stem cells obtained may be used in any of the aspects of the invention that utilise stem cells. They may also be used in any of the other applications of stem cells. They may, for example, be used in the generation of non-human chimeric animals and hence transgenic non-human animals.

It is shown here that embryonic stem cell like cells can be generated from adult stem cells using RNA extracted from embryonic stem cells. It is also shown that other differentiated adult tissue can be differentiated into stem cell like tissues when subjected to various stem cell-derived RNA fractions.

The invention provides cells obtained using the above methods. The cells may be provided in some cases as frozen aliquots in suitable receptacles. The invention also provides cell extracts of the cells.

In Vitro Methods for Inducing the Differentiation of Stem Cells

The invention also provides methods for inducing the differentiation of stem cells in vitro. The differentiation is achieved by providing the cell with a RNA sequence comprising a RNA extractable from the cell type that it is desired to differentiate the stem cell into. The RNA may be extractable or extracted from cells comprising said desired cell type(s). In particular the invention provides a method of inducing in vitro totipotent or pluripotent stem cells of a stem cell line or obtained from a tissue of an animal or plant to differentiate into one or more desired cell types, which comprises providing isolated RNA comprising a RNA sequence extractable from tissue or cells comprising said desired cell type(s) to a cell culture of said stem cells under conditions whereby the desired differentiation of said stem cells is achieved.

Any stem cell may be used in the methods, including any of those mentioned herein. In a preferred embodiment, the stem cells to be differentiated may be obtained using the methods of the invention to reverse the differentiation of differentiated cells to provide the stem cells. In cases where the differentiated cells obtained are intended for use in the treatment of a subject, or in the manufacture of medicaments to treat a subject, the stem cells may originate from the intended recipient. In some cases the stem cells may originate from a recipient who has a genetic defect and preferably the genetic defect may have been corrected or ameliorated in the stem cells in such cases.

The RNA may be provided to the target stem cells using any of the methods discussed herein.

The stem cells may be induced into any desired cell type including any of those mentioned herein. In a preferred case the stem cell will be differentiated into a stable terminal differentiated cell type. A terminal differentiated cell type may generally be considered as one that does not naturally differentiate to give any other cell type and is typically at the end of a lineage. In some cases the stem cell may be differentiated into an intermediate cell between the stem cell and the terminal cell of the lineage. Such intermediates may have some degree of proliferative capacity.

The differentiated cell may be one of an organ or tissue such as the liver, spleen, heart, kidney, skin, gastrointestinal tract, eye, or reproductive organ. In a preferred embodiment the differentiated cell type may be one that is missing, present in reduced number or defective in a particular condition. The condition may be any of those mentioned herein and include injury, degenerative disease or a condition resulting from a genetic disorder. In a particularly preferred embodiment the differentiated cell may be an islet of Langerhans cell as the resulting cells can be used to treat diabetes. In another case the differentiated cell may be one of the central nervous system that can be used to treat a disorder or injury of the nervous system and particularly a disease of the brain or a spinal cord injury. In a preferred embodiment bone marrow stromal cells may be differentiated into neuronal cells.

In some cases the stem cell that is differentiated may be a pluripotent, but not totipotent, stem cell. In such cases the stem cell may, for example, be differentiated into a cell type that the stem cell is known to differentiate into in the organism it is isolated from.

In a preferred embodiment, bone marrow stromal stem cells may be differentiated into neuronal cells. In particular, they may be differentiated into neuronal cells expressing neuronal marker proteins (NeuN). Typically, the bone marrow stem cells may be differentiated into neuronal cells by providing an isolated RNA comprising RNA extractable from one or more types of brain cells or brain cell lines. In some cases the RNA may comprise a RNA extractable from brain tissue and in particular it may comprise a RNA extracted from a brain tissue. In a particularly preferred case the RNA may comprise RNA extractable from cortical neurones or a cortical neurone cell line. In some cases RNA extractable from neurones found in other locations than the brain may be employed or from cell lines derived from such neurones.

In another preferred embodiment, bone marrow stem cells may be induced to differentiate into muscle cells and in particular into skeletal muscle cells. Typically the RNA sequence provided will comprise a RNA extractable from or extracted from muscle cells or muscle cell lines and in particular from muscle stem cells.

In another preferred embodiment, pre-treatment of bone marrow stem cells with spine derived RNA dramatically improved the efficacy of stem cell treatment in an established model of progressive neurodegenerative disease. Typically in this embodiment, the RNA sequence provided will comprise a RNA extractable from or extracted from spine cells or other cells in the peripheral nervous system. This methodology may also involve the administration of such RNA in vivo to influence the proliferation, migration and functional integration of stem cells in situ.

In another preferred embodiment, pre-treatment of stem cells with brain derived RNA has been shown to increase their proliferation, migration and functional integration into recipient nervous systems. Further, RNA sourced from a more immature developmental stage, at an active cell generative stage, appears to have a more profound effect on stem cell stimulation and their consequent ameliorative effect in both age and disease related damage. This methodology may also involve the administration of such RNA in vivo to influence the proliferation, migration and functional integration of stem cells in situ.

The invention provides cells obtained using the above methods. The cells may be provided in some cases as frozen aliquots in suitable receptacles. The invention also provides cell extracts of the cells.

In some cases the stem cells may be present in or on a structure such as a support, membrane, implant, stent or matrix when they are differentiated or alternatively the differentiated cells may be added to such a structure. The structure may then be used in the manufacture of a medicament for treating any of the conditions mentioned herein. Mixtures of different differentiated cell types may also be made, for example, to mimic populations occurring together in vivo.

In one preferred embodiment the in vitro method may comprise:
providing a stem cell population and culturing it in vitro according to established protocols;
providing RNA extracted from a desired target tissue type (for example neurones, glia, muscle or any of the differentiated cell types mentioned above) to the stem cells; and
maintaining the cells in culture.

In a further preferred embodiment the in vitro method may additionally comprise the step of
extracting RNA from a desired target tissue type (for example neurones, glia muscle or any of the differentiated cell types mentioned above).
   In these embodiments of the invention, the RNA may be preferably be provided to the stem cells either 1) as naked RNA extract 2) via liposome mediated transfer 3) by electroporation of recipient cells or other established methods.

Preferably the resulting differentiated cells may then be formulated into a medicament which can be administered to a subject by an appropriate route such as via the sub-cutaneous, sub dermal, intra-venous or intra peritoneal routes.

General

The term “comprising” encompasses “including” as well as “consisting” e.g. a composition “comprising” X may consist exclusively of X or may include something additional e.g. X+Y.

The word “substantially” does not exclude “completely” e.g. a composition that is “substantially free” from Y may be completely free from Y. Where necessary, the word “substantially” may be omitted from the definition of the invention.

The term “about” in relation to a numerical value x means, for example, x±10%.

Modes for Carrying Out the Invention

The following Examples illustrate the invention.

### EXAMPLE 1

### Production of Neural and Muscle Cells From Bone Marrow Stromal Stem Cells

Marrow Harvest and Culture.

Bone marrow stromal (mesenchymal) stem cells were obtained from adult Sprague Dawley rats. The technique is based upon the protocol of Owen and Friedenstein (1988), and represents a typical established adult stem cell source suitable for expansion in vitro. Briefly, after schedule one killing (cervical dislocation), tibia and femora were excised within 5 minutes of death. All connective and muscular tissue was removed from the bones and all further procedures were conducted under sterile conditions.

Marrow was expelled from the bones by flushing the bones with media (α-MEMS—Gibco Invitrogen Co. UK) containing 10% foetal calf serum, and 1% penicillin/streptomycin. Flushing was achieved by inserting a 25-guage needle attached to a 5 ml plastic barrel into the neck of the bone (cut at both distal and proximal end) and expelling 2 ml of media through the bone. The media and bone marrow sample were collected in sterile universal containers. Bone marrow cells were subsequently dissociated by gentle trituration through a 19-guage needle approximately 10 times. One ml of aspirate was then placed in six well plates (SLS Ltd. UK). Two ml of fresh α-MEMS was then added to each well giving a plating density of approximately 12,000-15,000 cells per ml. Plates were then incubated at 37° C., in 5% CO₂ in air and left undisturbed for 24 to 48 hours (Harrison & Rae, 1997).

Following this time period, marrow derived stem cells were isolated from non-plastic adherent cells by aspirating the culture media from the plate. Plastic adherent marrow stromal stem cells remained, and were supported by the addition of 2 ml of fresh α-MEMS (10% foetal calf serum and 1% penicillin/streptomycin). New media was applied every 48 hours until the plate was confluent with colony forming units (CFU's) confirmed by microscope analysis (Owen & Friedenstein, 1988, supra). Under optimal conditions this required 5 to 7 days at 37° C. Resultant cells were confirmed as stromal stem cells morphologically and immunohistochemically.

RNA Procedure

Brain homogenate was prepared and RNA separated using a RNA commercial separation kit or standard phenol based procedures. In the initial experiment, RNA was prepared by a cold phenol extraction method based on the method of Kirby (1956). Brains were freshly dissected from eight freshly killed rats. Eight grams of brain, excluding the cerebellum, was weighed and 5 ml of phosphate buffered saline (PBS) was added. The mixture was homogenised in a glass Teflon homogeniser for approximately 4 minutes. An equal volume of 95% saturated phenol was added. The resultant solution was left at room temperature for 15 minutes then centrifuged at 18,000 rpm in an ultra centrifuge for 30 minutes. The aqueous phase was retained and brought to a concentration of 0.1M MgCl₂ by the addition of 1M MgCl₂. Two volumes of ethanol were then added and precipitation was allowed to occur for approximately 30 minutes. A final spin at 6,000 rpm for 15 minutes produced a RNA rich precipitate, which was retained and stored under ethanol. Resultant RNA was air dried and dissolved in 6 ml of fresh media as defined above.

One ml of media containing the RNA was added to each well of confluent bone marrow stem cells for 24 hours. After 24 hours the RNA media was removed and replaced with fresh media. Cells were observed for phenotypic change every 12 hours.

Furthers cells were subjected to immunohistochemical analysis to confirm that the RNA induced in the bone marrow stem cells was a neuronal phenotype. This was achieved by testing treated cells for the expression of a neuronal marker NeuN. The results obtained are indicated in the Table below.

| Cells | Morphology | NeuN |
|---|---|---|
| Untreated cells | Retained CFU morphology | − |
| Brain RNA treated cells | Developed Neuronal type Morphology | + |

Examination of the cells showed the RNA induced change in cellular differentiation to a clear neuronal phenotype 24 hours after application of brain derived RNA. Untreated bone marrow stem cells retained the classic colony forming unit morphology. However, as early as 12 hours post-treatment the brain RNA treated stem cells showed typical neuronal and glial morphologies. Further, these cells expressed a commonly used immunochemical marker for neurones. Control cells did not. This change in phenotype survived passage (×3) and thus would appear a stable change in recipient stem cell differentiation. That donor tissue RNA was responsible for the change in stem cell differentiation was confirmed by subsequent experimentation in which the inductive effect of RNA was abolished by pre-treatment with RNaze, yet remained resistant to treatment of the donor brain RNA with trypsin, a potent protease.

The experiment was repeated using donor RNA, derived from skeletal muscle to confirm the specificity of the induced differentiation. It was clearly visible that the stem cells prepared as above and treated with muscle derived RNA (prepared using a commercially available kit, RNAzol), showed a stable differentiation change to muscle phenotype. This was confirmed by immuno staining with Phospholamban and Phalloidin. In the muscle study, the stem cells were exposed to muscle derived RNA (derived with a different RNA separation technique) via a different method of RNA delivery. RNA was delivered to the stem cells via liposomes prepared after the methodology of Felgner et al. (1987). Thus it can be concluded from these studies that the induction in stem cells is specific to the donor tissue source, and that the RNA can be added to the stem cells via a variety of techniques commonly employed to deliver nucleic acids to cells.

### EXAMPLE 2

### The Effects of Brain RNA Differentiated Stem Cells on Age Related Damage to the Rat Brain, Assessed by Spatial Learning and Memory Performance of Recipient Animals

Bone marrow mesenchymal stem cells were prepared in vitro as described above in Example 1. When the cells reached confluence, they were exposed to brain RNA (prepared as above) for 12 hours. Donor stem cells were derived from a pigmented rat strain (Lister Hooded). Donor RNA and recipient animals were provided from a different rat strain (Sprague Dawley).

Recipient Sprague Dawley rats were ex-breeder male rats aged between 468-506 days. It is well established that such animals of advanced age cannot learn to locate a hidden platform in a water maze (Stewart & Morris, 1993; Bagnall & Ray, 2000). Experimental animals received a 0.5 ml intra-venous injection of brain RNA treated stem cells, equating to the product of one six well plate of brain RNA treated cells. Control animals received an equivalent amount of untreated stem cells. Briefly, cells were collected from plates, either treated (experimental) or untreated (control) by mechanically removing them from the plastic plates using a rubber policeman and collected, by aspiration, in culture media. Cells were concentrated via a 5 minute spin at 1000 rpm and brought to a concentration outlined above. All injection procedures were conducted blind. For both groups, injections were mediated via the tail vein.

Fourteen days after injection, the aged rats were assessed blind on a commonly used spatial learning task, the Morris water maze. Each animal received 3 swims per day over a 3 day period with an inter trial interval of 10 minutes (Stewart & Morris, 1993). Latency to find the platform on each trial was recorded for each animal. Each trial consisted of a 60 second swim. If after that interval the animal had not located the platform, it was gently guided to the platform by the experimenter. Upon reaching the platform, the animal was allowed 10 seconds to orient to its location prior to removal to the home cage. Learning is evidenced by a decrease in time to locate the platform over repeated trials.

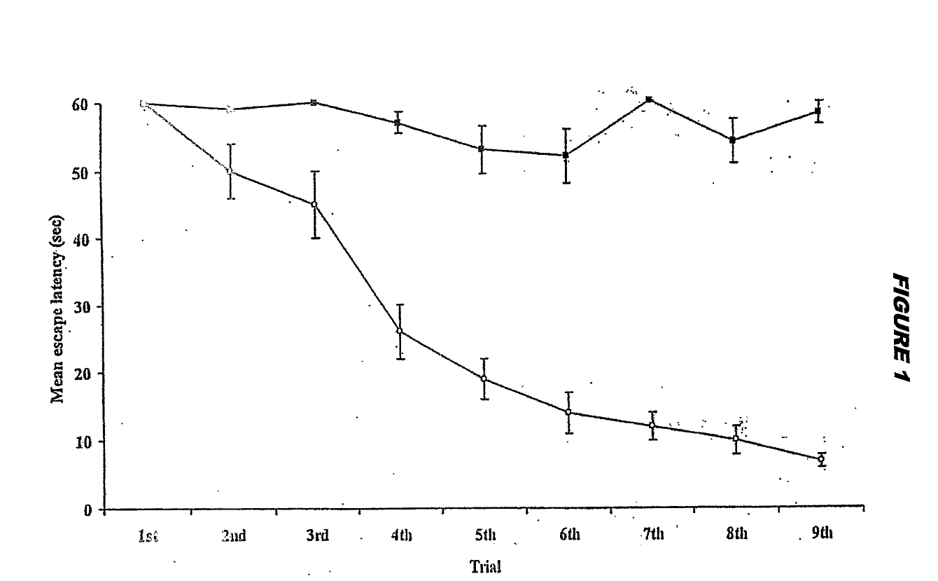

The results of the study are presented in FIG. 1. Control rats (n=9) receiving intra venous stem cells which had not been exposed to RNA, could not learn this task with no decrease in response latency over trials. However, the experimental animals receiving brain RNA treated stem cells showed a remarkable learning ability comparable to that of young rodents (p<0.0000000001). Two conclusions may be drawn from this study. First, RNA treated stem cells can significantly ameliorate age related deficits in spatial learning. Control untreated stem cells cannot. Second, it should be noted that donated stem cells were from a different strain of rat and recipient animals were not rendered immunodeficient. Thus, the results suggest that not only did the experimental group cells differentiate to appropriate neural tissue capable of functional improvement, they acquired an immunological status rendering them acceptable to the recipient. It should be noted that donor brain RNA was sourced from sibling animals to the recipients, yet donor cells were sourced from a different strain.

The results not only confirm that RNA differentiated stem cells can repair age related damage by restoring behavioural capabilities, but further that such treated cells acquire the immune characteristics of the donor RNA. This offers a strategy to change the immune profile of stem cell lines or stem cell banks to create differentiated cells with specific compatibility with the recipient.

### EXAMPLE 3

### In Vivo Stimulation of Resident Stem Cells Via Exogenous RNA Stimulated Differentiation, Migration and Integration

Given the powerful stimulatory effects of exogenous RNA on stem cells established in Examples 1 and 2, and the effects of these cells on repairing age related damage in a mammalian model, a further Example is given, establishing the effects of primary tissue derived RNA on host animal resident stem cells. To this end, neonate rats received an intraperitoneal injection of donor GFP-expressing crude bone marrow at age 1 day postnatal. Each animal received approximately 800,000 cells in a 0.2 ml injection. These foreign cells were readily integrated in host bone marrow and were observed to contribute to this biological environment. At age 90 days, GFP bone marrow grafted animals were randomly assigned to two groups.

Experimental animals received an injection of brain RNA, control animals received an injection of physiological saline. Experimental brain RNA was prepared as outlined in Example 1. Injection was conducted sub-cutaneously. Each animal received one whole brain equivalent of donor RNA in a 0.5 ml injection. Controls received an equivalent injection of physiological saline.

The results obtained showed a significant thickening of recipient cortex (p<0.0001) in experimental animals compared to control animals. Further, a significant number of differentiated neurones and glia in experimental animals showed expression of GFP indicating infiltration of resident bone marrow stem cells into the brain following application of exogenous brain RNA.

### EXAMPLE 4

### Induced Differentiation of Stem Cells Via Exogenous RNA Isolated From a Primary Cell Culture of Cortical Neurones

A purified culture of embryonic cortical neurones was established in the laboratory following the protocol of Saneto and deVellis (1987). Briefly, time mated Sprague Dawley female rats were sacrificed at day 16 of gestation. The abdominal area was sterilised with 70% alcohol and the uteri exposed. Uteri containing the embryos were then dissected free from the uteri and placed in a large 100 mm Petri dish. All the above procedures were conducted on a clean bench outside the sterile hood to prevent contamination. All further procedures were conducted under sterile conditions.

Intact uteri were then washed with physiological saline and transferred to another sterile Petri dish. Embryos were then dissected free from the uteri and placed in a new Petri dish for brain dissection. Brain tissue was exposed and gently removed with a spatula and cortices were dissected under a dissecting microscope. Meninges were then dissected clear in physiological saline. After cortices were processed, they were gently disrupted with repeated passage through a 10 ml glass pipette. The cell suspension was then passed through a Nitex 130 filter (mesh size 130 μm) and the filtrate centrifuged at 40 g. The pellet was then re-dispersed in serum free basal media (Saneto & deVellis, 1987, supra) and passed through Nitex 33 (mesh size 33 μm) and cells counted.

The suspension was supplemented with insulin (5 μg/ml) and transferrin (100 μg/ml) to form neurone-defined medium. Cells were seeded at a density of 1×10⁵ per well on 24 well culture plates pre-coated with polylysine (2.5 μg/ml). Cultures are reported as containing more than 95% neurones by immunological criteria of expressing the marker neurofilament protein, while not expressing the biochemical and immunological markers for astrocytes and oligodendrocytes (Saneto & deVellis, 1987, supra). Media was changed every third day post plating and cultures were maintained for 12 days prior to RNA extraction.

RNA was extracted from the primary cortical neurone cultures via a commercial kit (RNAzol) using the manufacturer's protocol. Resultant RNA was collected and redissolved in bone marrow culture medium (as defined in example 1) just prior to application to a confluent colony of rat bone marrow cells prepared as in Example 1. Each recipient bone marrow culture well received the total RNA extracted from one complete 24 well primary neuronal culture (although similar results were obtained a wide variety of exogenous RNA concentrations).

Bone marrow stem cells were examined microscopically 24 hours after application of exogenous RNA dissolved in media. Control bone marrow stem cells received an equal amount of RNAzol prepared bone marrow stem cell RNA.

Results showed all experimental stem cell wells produced clearly differentiated neurones, which stained positively for neuronal markers. No observable change in stem cell differentiation was found in the Bone marrow RNA treated wells. These results suggest that donor RNA from a purified cell source may induce highly specific stem cell differentiation.

The differentiation inducing effect of exogenous RNA fractions was sensitive to pre-treating the donor RNA with RNaze yet insensitive to trypsin. This suggests that RNA mediated the effect. These effects may be repeated using a wide range of RNA doses delivered exogenously by a variety of delivery methods and vehicles including liposomes or electroporation.

### EXAMPLE 5

### Retro-Transformation of Terminally Differentiated Cells Via Exogenous Application of RNA Fractions Obtained From Stem Cell Sources

Given the powerful and specific effects of RNA tissue extracts on stem cell differentiation in Examples 1 to 4, a final example of the technology is provided. Here, the donated RNA rich extract is obtained from cultured stem cells. Its ability to reverse differentiation is tested by exogenous application to terminally differentiated adult fibroblasts to investigate if recipient mature differentiated cells could be re-differentiated to stem cell character and behaviour via stem cell derived RNA fractions. The results obtained show that stem cell type tissue may be generated from differentiated tissue.

Adult rat (Lister Hooded) fibroblasts were obtained and maintained in culture conditions according to the protocol of Kawaja et al., (1992). A biopsy of skin (approx. 1 cm²) was placed into a sterile Petri dish containing phosphate buffered saline (PBS), pH7.4. The biopsy was then dipped (×3) in another dish filled with 70% ethanol then placed back in fresh PBS and cut into 1-2 mm pieces. These explants were placed into 60-mm tissue culture dishes pre-filled with 1 ml Delbecco's minimal essential medium supplemented with 10% f fetal bovine serum (FBS) and 0.1% glutamine. 10 units/ml of penicillin and 100 μg/ml streptomycin were also added. This culture was incubated with 5% CO₂ at 37° C.

After two days in such culture conditions, fibroblasts begin to migrate from the explant, at this stage an additional 2-3 ml of nutrient media was added.

When the plates reached approximately 90% confluence, they were passaged by incubating the cultures with 1-2 ml of trypsin solution and transferred to a 15-ml centrifuge tube, then centrifuged in a bench centrifuge for 10 minutes at room temperature. The supernatant was discarded and the pellet resuspended in 10 ml of culture medium. These cells were maintained in untreated 6 well plates seeded with 0.5 ml cell suspension in 2 ml of medium until confluence. At this time they could be further passaged.

Donor RNA was sourced from adult rat bone marrow mesenchymal stem cells maintained in culture as reported in Example 1 or from neural stem cells (neurospheres) cultured according to the protocol of Reynolds & Weiss (1992). All RNA rich extracts were prepared by RNAzol separation following the manufacturer protocol. Thus, two donor RNA fractions were obtained: 1) bone marrow stem cell RNA (BMS-RNA) and 2) neural stem cell RNA (NS-RNA). These fractions were dissolved respectively in fibroblast growth media at various concentrations from 0.75 μg/ml to 500 μg/ml and added to adult differentiated fibroblasts maintained in final culture wells for 5 days. Transformation of fibroblasts via stem cell derived exogenous RNA appeared across a wide range of doses.

In the results obtained, differentiated fibroblasts with no treatment of exogenous stem cell RNA showed no change in phenotype. 48 hours after RNA application, fibroblasts treated with an exogenous RNA dose of 25 μg/ml of either NS-RNA or BMS-RNA both showed a clear change in morphology. Recipient fibroblasts of NS-RNA formed floating spheres with the appearance and characteristics of neurospheres, from these neural phenotype cells began to radiate these could be easily identified as both neuronal and glial in morphology. Recipient fibroblasts of BMS-RNA, at for example 25 μg/ml, showed the classical bipolar shape of mesenchymal stem cells and were plastic adherent.

Subsequent experimentation showed these cells to be able to produce neurones and muscle tissues when further induced by exogenous RNA as described in Example 1. The retro-differentiation inducing effect of exogenous stem cell derived RNA fractions was sensitive to pre-treating the donor RNA with RNaze yet insensitive to trypsin. This suggests that the effect was mediated by RNA. These effects may be repeated using a wide range of RNA doses delivered exogenously by a variety of delivery methods and vehicles including liposomes or electroporation.

Thus, differentiated adult tissue can be retro-differentiated into stem cell like tissues when subjected to various stem cell-derived RNA fractions. The properties of the resulting cells reflect the donor stem cell morphology, behaviour and potential. Thus a novel and ethically less contentious way of obtaining both totipotent and pluripotent stem cells for a variety of applications in regenerative medicine is provided.

### EXAMPLE 6

### Comparison of Spine RNA Treated Bone Marrow Stem Cells with Undifferentiated Bone Marrow Stem Cells in an Animal Model of Motor Neurone Disease

The SOD 1 mouse is a well-established animal model of human motor neurone disease. These transgenic animals begin to show hind limb paralysis at 70-90 days with aggressive loss of motor neurones and death at 120-135 days.

Thirty animals were used in the study. All were confirmed to express the SOD 1 genotype. Animals were randomly assigned into three groups as follows:
(i) group 1—bone marrow stem cells incubated with spine RNA;
(ii) group 2—bone marrow stem cells only; and
(iii) group 3—PBS injection.

Donor bone marrow stem cells were harvested and cultured as described in Example 1. Spine RNA was prepared from freshly dissected adult C57/B1 mice using the Kirby protocol described in Example 1. Stem cells to be used in group 1 were incubated with spine RNA for 5 hours (250 μg/ml), washed twice in fresh media, and then concentrated for injection at approximately 90,000 cells per animal in 0.1 ml. Stem cells prepared for injection in group 2 were maintained in culture with no exposure to RNA and given 5 hours equivalent exposure to fresh media.

Recipient animals in each group received an injection via the tail vein. Injections were mediated using a 30 G needle. Injections were performed on recipient animals between the ages of 72 and 86 days at which time all animals showed hind limb paralysis. The number of animals surviving in each condition was recorded daily. Further limb movement was assessed weekly on a simple run test to observe hind and forelimb function.

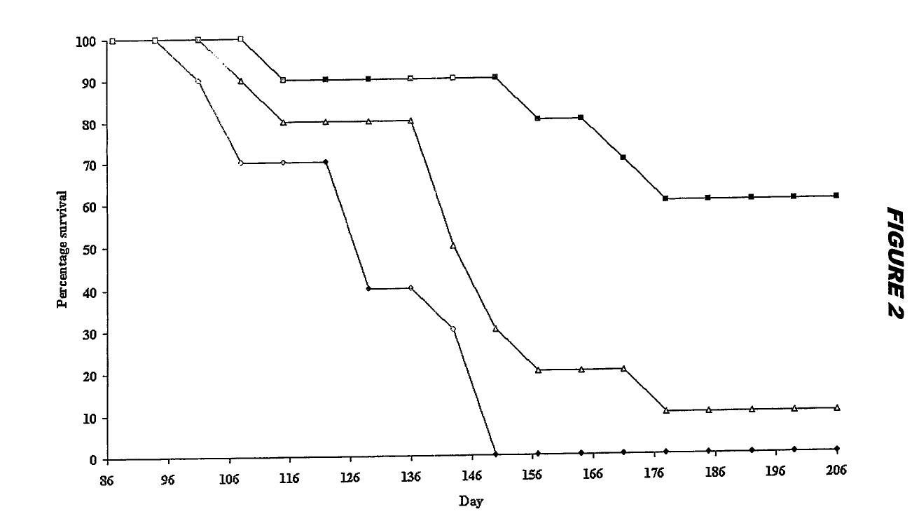

The results of this study are illustrated in FIG. 2. Pre-treatment of stem cells with spine derived RNA dramatically improved the efficacy of stem cell treatment in an established model of progressive neurodegenerative disease. Untreated bone marrow derived stem cells did have some effect but the novel step of pre-differentiating stem cells with RNA dramatically improves the effect. It is further noted from this example that all surviving animals in the RNA stem cell group (6) and the survivors in the stem cell only group (1) had complete recovery of pre-treatment paralysis and the treatment prevented further evolution of this normally progressive disease.

### EXAMPLE 7

### Effects of RNA Donor Tissue Age and Developmental Stage on Stem Cell Migration, Integration and Repair

Having established the effects of donor tissue derived RNA on stem cells in a variety of applications, a further example is provided investigating the effects of donor tissue developmental stage, prior to RNA extraction, on stem cell proliferation, migration and integration into host tissue.

Bone marrow stem cells were harvested and cultured as outlined in Example 1 from Tau-GFP-expressing mice. Recipient animals (N=24) were 254-299 day old C57/B1 mice randomly assigned to three recipient groups (n-8). Cultures of stem cells were randomly allocated to three conditions for RNA treatment prior to injection:
(i) group 1 foetal (E15) brain RNA+stem cells;
(ii) group 2 adult (90 day) brain RNA+stem cells; and
(iii) group 3 stem cells+no RNA.

RNA was extracted using the Kirby method as detailed in Example 1 and the appropriately sourced RNA detailed above was dissolved in media at a concentration of 200 μg/ml. Each well of recipient stem cells was incubated in 2 ml of fresh media supplement with 1 ml of RNA containing media (groups 1 & 2) or 3 ml of fresh media only (group 3) for 12 hours. Cells were then washed twice and concentrated for injection at approximately 40,000 cells in 0.3 μl of fresh media. Recipient animals were anaesthetised and cells were injected using stereotaxic guidance into the left lateral ventricle of the brain. Twenty days after surgery all groups were assessed on a mouse Morris water maze using the same training protocol as reported for rats reported in Example 2. Mice at this age show similar spatial learning deficits to old rats using this training methodology. After training, recipient rats were sacrificed and brain tissue was examined for cortical thickness and fluorescent microscopy to assess survival, proliferation and migration of GFP-expressing cells.

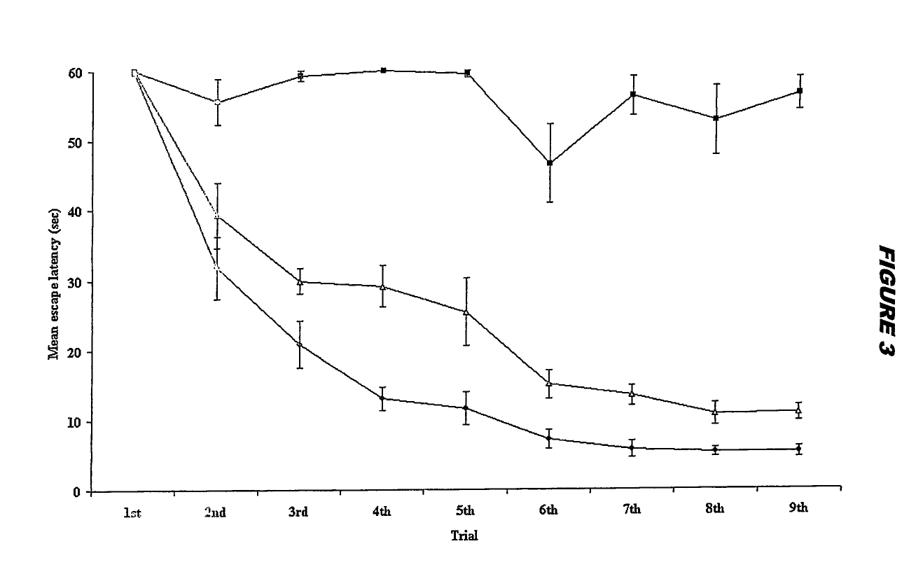

Behavioural results are presented in FIG. 3. Animals in both groups 1 and 2 showed excellent learning on the Morris water maze when compared to animals in group 3. This further shows the stimulatory effect of exogenous RNA treatment on stem cells in repairing age related brain damage (see Examples 2 and 6). Further, the foetal RNA+stem cell group showed significantly (p<1×10⁻¹⁰) faster acquisition of the task than the adult RNA+stem cell group. These data indicate that RNA sourced from a developmental stage when extensive neurogenesis is occurring may have a more profound effect when used to treat stem cells for tissue repair. Examining cortical thickness further supported this conclusion.

Measurement of cortex thickness in 20 identical anatomical cross sections in each animal showed a significant difference between the adult RNA+stem cells recipients and the stem cell only group (p<1×10⁻⁵), this confirms similar rat data (see Example 3). However, the cortex measures in the foetal RNA+stem cell group was also significantly thicker than the adult RNA group. Optical examination under fluorescent microscopy showed that the adult RNA+stem cell group had GFP-expressing cells extensively throughout the injected and contralateral hemispheres. However, foetal RNA+stem cell animals had approximately 30% more cells than the adult RNA group throughout the cortex of both hemispheres. GFP-expressing cells in the stem cell only group was predominantly located around the lower margins of the injected lateral ventricles and the olfactory bulbs. Only occasional cells were located in the ipsilateral cortex.

It can be concluded from this study that pretreatment of stem cells with brain derived RNA increases their proliferation, migration and functional integration into recipient nervous systems.

Further, RNA sourced from a more immature developmental stage, at an active cell generative stage, may have a more profound effect on stem cell stimulation and their consequent ameliorative effect in both age and disease related damage.

### EXAMPLE 8

### A Comparison of the Stimulatory Effects of Adult Stem Cell Derived RNA on Endogenous Neural Stem Cells and their Activity

Evidence provided in Example 3 shows that exogenous RNA had a stimulatory effect on resident bone marrow stem cells in restoring age related behavioural deficits. It is also described (Example 5) that stem cell derived RNA can influence differentiated tissues. This Example investigates if direct injection of bone marrow stem cell derived RNA can stimulate endogenous repair mechanisms to ameliorate age related behavioural deficits. Various endogenous neural repair processes are now known, including direct neurogenesis mediated by neural stem cells, but also secretion of survival factors from stem cells, which may influence damaged differentiated tissues.

Bone marrow stem cells were harvested and cultured in vitro as described in Example 1. Confluent cultures were then selected for RNA extraction. RNA extraction was mediated using a commercial product RNAzol following the manufacturer's instructions. Resultant bone marrow RNA was dissolved in PBS (200 μg/15 μl) ready for injection into recipients.

Recipient Sprague Dawley rats were ex-breeder males aged between 433 days and 570 days. Due to profound age related damage to the CNS such animals cannot learn or recall the Morris water maze task. Recipients were matched for age into two groups of 10 animals:
(i) group 1—received a 15 μl injection of stem cell RNA; and
(ii) group 2—received a 151 μl injection of stem cell RNA treated with RNaze (see Example 1).

Injections were made under anaesthesia into the right lateral ventricle under stereotaxic guidance. Briefly, recipient rat was anaesthetized, head shaved and placed in a stereotaxic frame. Skin was swabbed with 100% alcohol and the skull exposed by longitudinal incision. A 1.5 mm wide hole was drilled 1.5 mm anterior to the bregma and 1.5 mm lateral to the midline. The visible dura was cut with the tip of a 30 G hypodermic needle. The loaded cannula was lowered into the lateral ventricle via stereotaxic guidance and the contents ejected in 5 μl steps. The cannula was left in place for 2 minutes before removal and the incision closed with suture.

Fourteen days after injection, the aged rats were assessed blind on the Morris water maze as described in Example 2.

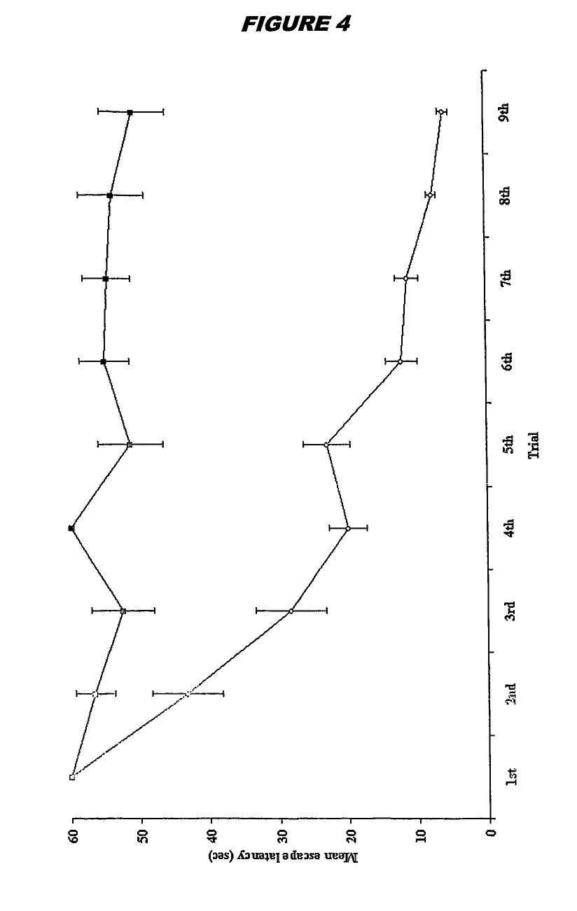

Results of this study are presented in FIG. 4. Control rats receiving deactivated stem cell RNA (RNaze treated) could not learn the task. There was no decrease in response latency over trials. However, the stem cell RNA treated animals all learned the task and were comparable in performance to young rats.

The stem cell derived RNA had a significant (p=1.28×10⁻⁴⁵) effect on stimulating endogenous repair mechanisms in the aged recipient brain. This may have been mediated by stimulation of resident neural stem cell neurogenesis per se or by increased production of secretory molecular products involved in tissue repair.

This experiment has also been replicated with a similar stimulatory effect using foetal (E12) derived whole brain RNA injected at a dose of 125 μg/μl (n—8) and a PBS injected control (n=8). Foetal RNA injected animals performed significantly better than control (p<1×10⁻⁵). This replication indicates that RNA prepared from developmental stages known to show increased stem cell activity may also be used to stimulate endogenous repair mechanisms.

### EXAMPLE 9

### The Effects of Foetal Brain Extracted RNA on Damaged Brain Tissue In Vitro

The results of the two studies in Example 8 suggests that exogenous RNA sourced from stem cell active tissues, or stem cell derived RNA, may influence not only endogenous stem cells but may also influence resident differentiated cells. This is also shown in Example 5. The current description investigates the effects of foetal brain derived RNA on adult brain cortex cells placed in vitro.

It is well established that foetal neurones survive in tissue culture, however adult cortical neurones do not survive well. The principal reason for this is the damage suffered during initial cell preparation and plating. The trauma of dissociation is known to produce irreparable damage. It was hypothesised that RNA from an actively developing (foetal) tissue may repair such damage and enhance the survival of these cells.

RNA was extracted from 3 g of fresh foetal (E18) cortex using the Kirby protocol described in Example 1.

Adult neural tissue was cultured via the technique described in Example 4 (Saneto & deVallis, 1987). This protocol produces excellent cultures of foetal cortical neurones, however adult cortex preparations do not survive using this method. Source cortex was dissected from 48 day old Sprague Dawley rats and plated at a density of approximately 1×10⁵ into 24 well plates. 96 wells were thus prepared. 24 hours after plating, all wells were observed to have large populations of dead, necrotic and dying cells. 12 wells per 24 well plate were treated with 150 μg of foetal brain RNA dissolved in the neurone culture media. The control wells each received fresh culture media. Cells were left undisturbed for a further 48 hours then all wells received a media change with fresh media. Media changes were repeated every 3 days. Cells were observed every 24 hours.

An initial observation at 24 hours post media change showed that all control wells were dead. No viable cells remained, clumps of floating debris were observed and a dense coating of dead material was found at the bottom of all control wells. All control wells were found to have cloudy discoloured media indicative of dead cultures. Experimental wells appeared in better health but still contained some dead material. Viable cells were, however, visible.

After 72 hours (second media change) all control wells were dead (and disposed of). Experimental wells contained cell debris, which was removed with the media change, however in all wells some viable cells remained attached to the plate. Visible from many cells were small neurite outgrowths and clear neural morphology.

After 96 hours all experimental wells had flourishing neurones many with visible axon and dendrite structures. 17/48 (35%) wells showed extensive cell contact and connectivity.

After 120 hours all experimental wells contained extensive cell populations showing both neurone and glia morphology. Extensive neural networks were evident in all wells.

Cells were maintained for a further 30 days and expressed neural morphology throughout.

This Example shows a novel methodology for the culture of adult neural tissue. Furthermore, it illustrated that RNA extracted from a stem cell rich foetal tissue source has a profound rescue effect on damaged cells. This suggests a novel approach to tissue repair and regeneration via foetal or cultured stem cell RNA deliverable via a variety of methods to aged, diseased tissue or intractable wounds or trauma.

### EXAMPLE 10

### The Use of Rat Embryo RNA to Enhance Stem Cell Involvement in Tissue Regeneration

Adult mammals, including human beings, have poor regenerative abilities in many tissues and organs compared to foetal stages, which often have extensive regenerative abilities. Two major factors associated with this loss of regenerative ability are scar tissue formation and loss of secretory molecules that recruit new cells to injured tissues. While many laboratories have reported the integration of injected stem cells into damaged tissues, this has been on a relatively small scale. It could be hypothesised that if the signalling mechanisms of the foetal stage could be recapitulated in the adult, this would improve the ability of stem cells to effect manor regeneration of structures which show little or no repair. This would include old established injuries with associated scaring which is known to inhibit stem cell migration, integration and repair potential. The methodology used is co-injection of whole embryo RNA with stem cells. The example provided illustrated the complete regeneration of an established ear punch hole lesion in adult rats following injection of whole rat embryo RNA and bone marrow stem cells.

15-day old foetuses were dissected from the uteri of time-mated Lister Hooded rats. Foetal tissues were disrupted mechanically by a Turex homgenizer in cold PBS. RNA was extracted using the Kirby protocol described in Example 1.

Bone marrow stem cells were cultured as described in Example 1 and concentrated for injection as described in Example 7.

The injury model involved 18 male Lister Hooded rats aged between 137 and 149 days at time of injection. All rats received a 1.5 mm hole punch injury to the left ear at 30 days prior to injection date to model an old established injury. Rats at this age do not regenerate ear tissue.

Experimental animals (n=6) received a tail vein injection of 800 μg of embryo RNA dissolved in 0.3 ml of PBS. One hour later, the animals received a second injection of approximately 2×10⁵ bone marrow stem cells suspended in 0.3 ml of α-MEMS culture media. Control animals (n=6) received an initial tail vein injection of approximately 2×10⁵ bone marrow stem cells followed by a econd injection of 0.3 ml PBS 1 hour later. A further group, no treatment controls (n=6), were ear lipped but received no treatment.

Animals were observed daily for any signs of regeneration of ear injury. Results showed no evidence of tissue repair or remodelling in the no treatment control group. Similarly, the stem cell only injected controls also failed to show any evidence of repair other than a slight inflammatory response lasting 17 hours in one animal around the site of the injury. The experimental animals treated with a combination of embryo RNA and stem cells showed complete closure of the injury in all animals between 6 and 9 days post injection. In 5 of the 6 experimental animals there was complete remodelling of the injury to the extent that there was no visible scar or evidence of the original lesion. Animal 3 showed complete closure of the lesion but a visible skin covered depression remained.

The results clearly show that stem cell mediated tissue repair and regeneration can be dramatically improved by co-injecting embryo derived RNA fractions with the stem cells. It is clear, from this example and other similar studies by the present inventors, that the embryo RNA alters the host tissue environment around the tissue to signal injected stem cells to the damaged area. Further, the established scarring of the injury was similarly altered to provide a permissive environment for stem cell infiltration and subsequent repair of the lesion. With such co-treatment, stem cells are recruited to the damaged tissues and can reverse the damage once in location by regeneration of the relevant tissue types. Of great significance is the fact that the damage model used in this example is an old well established injury which stem cell injection alone cannot repair. This method provides a novel method of improving the efficacy of any potential stem cell therapy. Similar results have also been found using RNA extracted from foetal tissue maintained in tissue culture and injected up to 48 hours prior to stem cell injection. Longer intervals have not yet been investigated. Similarly, simultaneous injection of the RNA with stem cells achieves a similar major regeneration of damaged tissue. It is postulated that the embryo RNA re-creates the permissive regenerative environment and signalling environment of the foetal period.

### EXAMPLE 11

### Generation of Rat Embryonic Stem Cell-Like Cells From Adult Rat Bone Mesenchymal Stem Cells

While much emphasis has been placed on the plasticity of adult stem cells in many research laboratories, others consider embryonic stem cells to offer the most promise in the future of regenerative medicine. Embryonic stem cells have several practical disadvantages such as the ethics of generating embryonic stem cells, contamination of cell lines or availability of suitable cells. This example seeks to use embryonic stem cell extracted RNA to convert adult bone marrow stem cells to an embryonic stem cell-like cell.

Isolation, growth and maintenance of rat embryonic stem cells (RESCs) was carried out following the protocols of Fandrich et al. (2002) and Ruhnke et al. (2003). Briefly, RESCs were isolated from the dissociated inner cell mass of 4 to 5 day old blastocysts derived from time-mated Sprague Dawley rats. Embryonic stem cells were maintained on a feeder layer of mitomycin-treated embryonic fibroblasts. Culture media consisted of high-glucose Dulbecco's modified Eagles medium, 10% heat inactivated foetal bovine serum, 1% 200 mM L-glutamine, 1% penicillin/streptomycin solution (50 IU/50 μg), insulin (0.09 mg/ml), 1,000 U/ml LIF and 5 ml nucleoside solution (as reported in Ruhnke et al. 2003).

These cells grow in distinctive smooth round clumps and stained positive for alkaline phosphatase, a commonly used ES marker.

RNA was extracted from these RESCs via RNAzol prep following the manufacturer's instructions. Adult bone marrow stem cells were cultured as reported in Example 1 in 6 well culture plates. Each confluent well was assigned either experimental (n=12) or control (n=12). Experimental wells received 150 μg of RESC RNA in 3 ml of bone marrow culture media (see Example 1) at a routine media change. Control animals received 3 ml of bone marrow culture media. After 24 hours there was a noted change in morphology of some of the cells in the experimental wells. The colony forming units, typical of bone marrow mesenchymal stem cells appeared disrupted and large numbers of aggregated smooth round clumps of cells appeared floating in the media. Their morphology was reminiscent of the RESC cultures. No such structures appeared in the control wells. These floating aggregated structures were aspirated with the media and placed onto feeder layers in RESC media as described above and maintained in long term culture. Over 60 days they retained their floating round aggregate morphology. After 60 days in culture these cells stained positive for alkaline phosphatase, the ES marker. Control well media was also aspirated and placed in identical wells conducive to RESC culture, no aggregated floating structures emerged.

This experiment suggest a novel method for generating embryonic stem cell like cells from adult stem cells with fewer ethical issues to address.

### EXAMPLE 12

### In-Vivo Injection of Muscle RNA From Exercise Tolerant Rats Induces Exercise Tolerance in Sedentary Rats

Exercise is known to be beneficial to muscle anatomy and physiology. During repeated exercise micro damage to skeletal muscle induces both stem cell activity and changes in muscle cell biology. Such changes facilitate an increased tolerance for exercise with practice.

RNA extracted from hind limb muscles from exercised rats was injected to sedentary animals to investigate the effects of such treatment on recipient animal performance during heavy exercise. The exercise task involved running on a revolving drum. Rats readily learned to stay on the apparatus by running at an appropriate speed dictated by the revolution speed of the drum. As the animal tires and stops running it falls into a plastic bin filled with shredded paper. Once running skill had been perfected, animals would happily run on the apparatus until exhaustion. After a period of initial training on the apparatus, run time was recorded as a measure of exercise tolerance.

Experimental Donor rats (n=10) were trained daily on a suitable exercise regimen as follows:

Week 1—Animals were given 5 trials per day (10 minutes) with inter-trial interval of 1 hour. The revolution speed was set at 15 mm/sec. If the animal fell, it was placed back on the drum for the full duration of the trial. All animals mastered this motor skill readily over this orientation week.

Week 2—Animals were given 5 trials per day with an increased speed of 37 mm/sec with a 1-hour inter trial interval. If an animal fell, it was immediately placed back on the apparatus. Each trial was of 15 minutes duration.

Week 3—Animals were given 1 trial per day at the same run speed but run until the first fall.

Week 4—Animals were given 1 trial per day to first fall criterion at a run speed of 97 mm/sec.

Control Donor rats (n=10) were not exposed to the exercise apparatus and remained in their home cage throughout the 4-week exercise period.

Both groups of donors were sacrificed at the end of week 4 and hind limb muscles dissected. RNA was extracted by the method outlined in Example 1. RNA was then stored in 900 μg doses ready for injection.

Recipient animals (n=20) were divided into two matched groups. All recipient animals received an orientation week of training on the apparatus as described in donor week 1 training. They received no further conditioning.

One day after last orientation trial recipient rats received 900 μg of RNA dissolved in 0.3 ml of PBS (IV) into the tail vein. Experimental recipients received exercised muscle RNA, control animals received un-exercised RNA.

One-week post injection all rats received a run trial as follows: 5 minutes gentle running at 15 mm/sec. All rats balanced and ran comfortably in this session. After five minutes balance trial, the speed was increased to 97 mm/sec and the duration to falling off/jumping off was recorded as a measure of exercise tolerance.

There was a clear difference between the two groups. Recipients of non-exercised RNA showed a mean exercise time of 3.54 minutes. Recipients of muscle RNA from exercised rats showed a mean exercise time of 6.19 minutes.

The RNA extracted from the exercised animals enhanced exercise tolerance in recipient animals compared to controls. These preliminary data suggest that RNA may transfer exercise induced muscle enhancement to naïve muscle via in vivo application. This may provide a valuable therapeutic approach to various muscle degenerative diseases or a novel method to improve muscle mass in disease, ageing or age related pathology. Further, the technique may be of value in agriculture.

It will be understood that the invention is described above by way of example only and modifications may be made whilst remaining within the scope and spirit of the invention.

### REFERENCES

Bagnall, L. & Ray, S. (1999) Rat strain Differences on performance in the Morris water maze, Animal Technology. Vol. 50 (2). 69-77.
Dai et al. (2000) Biol Blood Marrow Transplant. 6(4). 395-407.
Fandrich, F., Lin, X., Chai, G. X. et al. (2002) Preimplantation-stage stem cells induce long term allogeneic graft acceptance without supplementary host conditioning. Nature Medicine. Vol. 8. 171-178.
Felgner et al. (1987) Proc. Nat. Acad. Aci. Vol 84. 7413-7417.
Gennaro (2000) Remington: The Science and Practice of Pharmacy. 20th edition, ISBN: 0683306472
Graham & van der Eb (1978) Virology 52:456-457.
Harrison, M A. & Rae, IF. (1997) General Techniques of Cell Culture. Cambridge Univ. Press. Cambridge.
Kawaja, et al. (1992) In Neural Transplantation: A practical Approach. IRL Press. Oxford.
Keown et al. (1990) Methods in Enzymology 185:527-537
Kirby, K. S. (1956) J. Biochem. Vol. 64. 405.
Kirshenbaum et al. (1999) Curr Opin Struct Biol 9:530-5.
Mansour et al. (1988) Nature 336:348-352 (1988)
Owen, M. & Friedenstein, A J. (1988) Stromal stem cells: marrow derived osteogenic precursors CIBA Foundation Symposium 136. 42-60.
Reynolds, B. A. & Weiss, S. (1992) Science. Vol. 255. 1707-1710.
Ruhnke, M., Ungefroren, H., Zehle, G., Bader, M., Kremer, B., & Fandrich, F. (2003) Long-term culture and differentiation of rat embryonic stem cell-like cells into neuronal, glial, endothelial and hepatic lineages. Stem Cells. Vol. 21. (4). 428-436.
Saneto, R. P. & de Vellis, J. (1987) Neuronal and Glial cells: cell culture of the central nervous system. In Turner, A. J. & Bachelard, H. S. (Eds.) Neurochemistry: A practical approach. IRL Press. Oxford.
Stewart, C. A. & Morris, R. G. M. (1993) The watermaze. In Sahgal. A (Ed.) Behavioural Neuroscience. A practical approach. Vol. 1. IRL Press. Oxford.
Tada, et al. (2001) Curr Biol. Vol. 1(19). 1553-8.

1. A method of altering a property of a cell towards a property of one or more desired cell types comprising providing isolated RNA comprising a RNA sequence extractable from cells comprising said desired cell type(s) to a population of cells under conditions whereby the alteration of the cell property is achieved.

2. A method according to claim 1, wherein said isolated RNA is provided to a cell population in a patient.

3. A method according to claim 1, wherein said property is phenotypic.

4. A method according to claim 1, wherein said property is a cell function.

5. A method according to claim 1, wherein alteration of said property involves a genetic transformation so that said population of cells acquires an altered, inherited genotype.

6. A method according to claim 1, wherein the alteration of the cell property is the differentiation of a stem cell to an adult specialized cell.

7. A method according to claim 1, wherein the alteration of the cell property is the reverse differentiation of an adult speacialized cell to a stem cell.

8. A method according to claim 1, wherein the alteration of the cell property is the differentiation of a specialized adult cell to an adult cell of a different specialty.

9. A method according to claim 1, wherein the alteration of the cell property is a change in immunological profile.

10. A method according to claim 2, wherein said method improves stem cell mediated repair in the patient.

11. A method according to claim 2, wherein said method induces stem cell mobilization, migration, integration, proliferation and/or differentiation in the patient.

12. A method according to claim 2, wherein said method effects repair of diseased cells, alters the genetic constitution of cells, induces specific cell types and/or cell fates, changes the immunological profiles of cells, and/or induces particular desired immune functions or properties.

13. A method according to claim 2, which additionally comprises the step of providing stem cells to the patient.

14. A method according to claim 13, wherein said step of providing stem cells is sequential to, simultaneous with, or separate to said step of providing the isolated RNA.

15. A method according to claim 1, wherein the isolated RNA comprises a RNA sequence that is extractable from cells of a different developmental stage than the developmental stage of the cells to be treated.

16. A method according to claim 1, wherein the isolated RNA comprises a RNA sequence that is extractable from cells of a more active cell generative stage than that of the cells to be treated.

17. A method according to claim 1, wherein the isolated RNA comprises a RNA sequence that is extractable from cells from an individual who shows immunity or resistance to a disease or condition.

18. A method according to claim 1, wherein the isolated RNA comprises a RNA sequence extractable from foetal cells, neonatal cells, juvenile cells or embryonic stem cells.

19. A method of inducing in vivo or in vitro totipotent or pluripotent stem cells of a stem cell line or derived from a tissue of an animal or plant to differentiate into one or more desired cell types, which comprises providing isolated RNA comprising RNA extractable from tissue or cells comprising said desired cell type(s) to a cell culture of said stem cells under conditions whereby the desired differentiation of said stem cells is achieved.

20. A method of inducing in vivo or in vitro totipotent or pluripotent stem cells of a stem cell line or derived from a tissue of an animal or plant to mobilize, migrate, integrate, proliferate and/or differentiate, which comprises providing isolated RNA comprising RNA extractable from tissue or cells comprising said desired cell type(s) to a cell culture of said stem cells under conditions whereby the desired differentiation of said stem cells is achieved.

21. A method according to claim 1, wherein said cells are stem cells.

22. A method according to claim 19, wherein said stem cells are selected from adult animal stem cells or an adult stem cell line; or embryonic stem cells or a stem cell line derived from such cells.

23. A method according to claim 20, wherein said stem cells are selected from adult animal stem cells or an adult stem cell line; or embryonic stem cells or a stem cell line derived from such cells.

24. A method as claimed in claim 22 wherein said adult stem cells are bone marrow stromal cells, haematopoietic stem cells or neuronal stem cells or a corresponding derived stem cell line.

25. A method according to claim 1, wherein said cells are human stem cells or a human stem cell line.

26. A method according to claim 25, wherein said cells are caused to differentiate into one or more stable terminal cell types.

27. A method according to claim 26, wherein the cells are genetically modified prior to differentiation.

28. A method as according to claim 25, wherein the cells are derived from the intended recipient of said desired cells.

29. A method according to claim 1, wherein said RNA comprises RNA extracted from tissue or cells of an individual different from the source of the cells to be treated, said extracted RNA being derived from a donor having an immunological profile compatible with the intended recipient of the desired cells.

30. A method according to claim 1, wherein a RNA extract is provided for uptake by the cells which is a whole tissue or whole cell RNA extract.

31. A method according to claim 1, wherein RNA-extractable from one or more types of brain cell or brain cell line is provided for uptake by cells.

32. A method according to claim 1, wherein the cells are bone marrow stromal stem cells and the isolated RNA provided comprises RNA extractable from one or more types of brain cell or skeletal muscle or a corresponding derived cell line of either.

33-36. (canceled)

37. A method of reversing in vitro the differentiation of differentiated cells of a cell line or obtained from the tissue of an animal or a plant to produce a desired type or types of totipotent or pluripotent stem cell(s) or stem cell line(s), which comprises providing isolated RNA comprising RNA extractable from the desired type(s) of stem cell or stem cell line to a cell culture of said differentiated cells whereby the desired reversal of differentiation of the differentiated cells into said type(s) of stem cell or stem cell line type(s) is achieved.

38. A method according to claim 37, wherein the stem cell is as defined in claim 22.

39. A method according to claim 37, wherein the differentiated cells are selected from skin cells, bone marrow cells and haematopoietic cells or a cell line derived from such cells.

40. A method according to claim 37, wherein the differentiated cells are fibroblasts or a fibroblast cell line and the RNA is extractable from bone marrow stem cells or neuronal stem cells.

41. A method according to claim 37, wherein the isolated RNA provided comprises RNA extractable from bone marrow stromal stem cells, neuronal stem cells or a stem cell line derived from either.

42. An in vitro method of producing differentiated cells, which comprises:
i) performing the method according to claim 37 to produce stem cells or a stem cell line from differentiated cells;
ii) performing the method according to claim 19 on the stem cells or stem cell line to produce differentiated cells.

43. (canceled)

44. Cells obtained by the method according to claim 1.

45. (canceled)

46. A method of screening for a RNA sequence capable of conferring a desired property from one cell type to another, the method comprising the steps of
a) extracting RNA from cells comprising a desired cell type;
b) separating the extracted RNA into different fractions;
c) providing a fraction to a test cell;
d) analyzing the test cells for an altered property possessed by the desired cell type from which the RNA was extracted;
wherein a fraction that confers the altered property onto the test cell is identified as comprising a RNA sequence capable of conferring the desired property.

47. The method according to claim 23 wherein said adult stem cells are bone marrow stromal cells, haematopoietic stem cells or neuronal stem cells or a corresponding derived stem cell line.

48. Cells obtained by the method according to claim 19.

49. Cells obtained by the method according to claim 37.

50. Cells obtained by the method according to claim 42.

51. An in vitro method of producing differentiated cells, which comprises:
i) performing the method according to claim 37 to produce stem cells or a stem cell line from differentiated cells;
ii) performing the method according to claim 19 on the stem cells or stem cell line to produce differentiated cells; and
iii) introducing a genetic modification into the stem cells.
